(19) United States
(12) Patent Application Publication
(10) Pub. No.: US 2025/0242076 A1
(43) Pub. Date: Jul. 31, 2025

(54) WOUND DRESSINGS

(71) Applicant: ConvaTec Limited, Flintshire (GB)

(72) Inventors: David Parsons, Flintshire (GB); Hannah Connor, Flintshire (GB)

(21) Appl. No.: 19/064,586

(22) Filed: Feb. 26, 2025

Related U.S. Application Data
(63) Continuation of application No. PCT/GB2023/052215, filed on Aug. 25, 2023.
(60) Provisional application No. 63/401,237, filed on Aug. 26, 2022.

(30) Foreign Application Priority Data
Sep. 9, 2022 (GB) 2213188.2

(51) Int. Cl. A61L 15/44; A61L 15/18; A61L 15/28

(52) U.S. Cl. CPC A61L 15/44; A61L 15/18; A61L 15/28; A61L 2300/102; A61L 2300/106; A61L 2300/606

(57) ABSTRACT

The invention provides an antimicrobial wound dressing comprising a wound dressing substrate comprising at least one species of Formula (I), wherein X is a halogen, and wherein the species is complexed with a non-polymeric counterion and/or complexed with the wound dressing substrate.

[I—I—X]⁻ Formula (I)

### TECHNICAL FIELD OF THE INVENTION

The present invention relates to molecular iodine-based antimicrobial wound dressings.

### BACKGROUND TO THE INVENTION

A variety of different dressings have been developed for treating different types of wounds, ranging from superficial grazes and cuts to more serious and problematic wounds such as burns and ulcers.

Wounds can often become infected, and it is advantageous for wound dressings to be designed to prevent, treat and/or ameliorate effects of such infections. There has been much interest in using antimicrobials to achieve this. Conventional methods have included applying antimicrobial compounds to wounds directly prior to dressing application. Recent efforts have turned to incorporating the antimicrobial as part of the wound dressing to improve ease of use, safety, sustained action, etc.

An antimicrobial receiving much interest in this field is iodine—the therapeutic and antimicrobial properties of iodine have been known for centuries; however, effective incorporation of iodine into wound dressings has been notoriously difficult to achieve. Further, the high reactivity of molecular iodine renders many incorporation methods used with other antimicrobials unsuitable for use with iodine.

Recent methods have focused largely on adding iodine into dressings as iodophors, often comprising an iodine-based ionic species complexed with another typically high molecular weight species. A particular iodophor that has found recent medical application is povidone-iodine (PVP-I) which is a complex salt of polyvinylpyrrolidone with triiodide ions. Another common iodophor is cadexomer iodine, which includes iodine complexed with a polymer produced by the reaction of dextrin with epichlorohydrin.

The antimicrobial activity of an iodophor present in a wound dressing is dependent on the amount of “free” /“active” iodine (i.e. molecular iodine that can be released into the dressing, or into or onto the wound) and on the active iodine release profile (e.g. rate of release). The use of iodophors in wound dressings has advanced the field of using iodine in wound treatment; however, this method is not without disadvantages. For instance, iodophors provide non-ideal active iodine loadings and release properties. Iodine release is often very quick and non-sustained, leading to rapid exhaustion of active iodine from the wound dressings.

There exists a need for improved iodine-based wound dressings. It is therefore an aim of embodiments of the present invention to address or ameliorate one or more problems of the prior art. In particular, it is an aim of embodiments of the present invention to provide wound dressings which have one or more of the following advantages:
Optimized iodine retention and release properties—controlled active iodine release from the dressing—minimal or no premature iodine release.
Good active iodine loadings.
An even active iodine distribution.
Sufficient antimicrobial and/or antibiofilm activity.
Safe to use and/or limited toxicity.
Ease of manufacture.

It is also an aim of embodiments of the present invention to overcome or mitigate at least one problem of the prior art, whether expressly described herein or not.

### SUMMARY OF THE INVENTION

According to a first aspect of the invention, there is provided an antimicrobial wound dressing comprising a wound dressing substrate comprising at least one species of Formula (I), wherein X is a halogen, and wherein the species is complexed with a non-polymeric counterion and/or complexed with the wound dressing substrate.

[I—I—X]⁻ Formula (I)

In some embodiments, the species of Formula (I) is complexed with a non-polymeric counterion. In some embodiments, at least 25% of the total number of species of Formula (I) are complexed with a non-polymeric counterion, or at least 50, 75, 80, 85, 90, 95, 96, 97, 98, or at least 99% of the total number of species of Formula (I) are complexed with a non-polymeric counterion. In some embodiments, 100% of the total number of species of Formula (I) are complexed with a non-polymeric counterion. The species of Formula (I) may be entirely complexed with a non-polymeric counterion and may not be complexed with the wound dressing substrate or with any other chemical entity. In such embodiments, the species may be held to a surface of or within the wound dressing substrate by intermolecular forces between the species and the substrate. The intermolecular forces may comprise one or more chosen from: London dispersion forces, dipole-dipole interactions, hydrogen bonding, and combinations thereof. The intermolecular forces may comprise one or more of the group consisting of: London dispersion forces, dipole-dipole interactions, hydrogen bonding, and combinations thereof. In some embodiments, the wound dressing substrate is fibrous and the species may be held between and/or within fibres of the substrate. The species may be present throughout fibres of the substrate. The species may be present throughout the length and/or diameter of fibres of the substrate.

The non-polymer counterion may be or comprise a simple counterion. The counterion may be or comprise an inorganic counterion, preferably an inorganic counter-cation. The counterion may be or comprise a metal counterion. In some embodiments, at least one counterion is independently chosen from: an alkali metal cation, an alkaline earth metal cation, a group III metal cation, a transition metal cation, an ammonium cation, an aromatic nitrogen-based cation, and combinations thereof. In some embodiments, at least one counterion is independently selected from the group consisting of: an alkali metal cation, an alkaline earth metal cation, a group III metal cation, a transition metal cation, an ammonium cation, an aromatic nitrogen-based cation, and combinations thereof. At least one counterion may be independently chosen from: ammonium, calcium, iron, magnesium, potassium, pyridinium, quaternary ammonium, sodium, copper, silver, hydrogen, and combinations thereof. At least one counterion may be independently selected from the group consisting of: ammonium, calcium, iron, magnesium, potassium, pyridinium, quaternary ammonium, sodium, copper, silver, hydrogen, and combinations thereof.

A non-polymeric counterion also excludes oligomeric counterions (such as dimeric, trimeric cations etc.) and counterions that are chemically associated with a polymeric species (e.g. a povidone-iodine complex, which is a complex salt of polyvinylpyrrolidone with triiodide).

In some embodiments, no greater than 95% of the total number of species of Formula (I) are complexed with a non-polymeric counterion, or no greater than 90, 80, 70, 60, 50, 40, 30, 20, or no greater than 10% of the total number of species are complexed with a non-polymeric counterion.

In some embodiments, the species of Formula (I) is complexed with the wound dressing substrate. In some embodiments, at least 25% of the total number of species of Formula (I) are complexed with the wound dressing substrate, or at least 50, 75, 80, 85, 90, 95, 96, 97, 98, or at least 99% of the total number of species of Formula (I) are complexed with the wound dressing substrate. In some embodiments, 100% of the total number of species of Formula (I) are complexed with the wound dressing substrate. The species of Formula (I) may be entirely complexed with the wound dressing substrate and may not be complexed with a non-polymeric counterion or with any other chemical entity. In embodiments in which some or all of the species is/are complexed with the wound dressing substrate, the species may be held to a surface of or within the wound dressing substrate by the complex formed between the substrate and the species. In some embodiments, the species may be held to a surface of or within the wound dressing substrate by both the complex formed between the substrate and species and by intermolecular forces between the species and the substrate.

The species may be complexed with at least one polymer of the wound dressing substrate. The species may be complexed with the wound dressing substrate by an ionic and/or covalent interaction, preferably an ionic interaction.

In some preferred embodiments, there is provided an antimicrobial wound dressing comprising a wound dressing substrate comprising at least one species of Formula (I), wherein X is a halogen, and wherein the species is complexed with the wound dressing substrate by an ionic interaction.

The species may be complexed with the wound dressing substrate via at least one cationic group of the substrate.

In some preferred embodiments, there is provided an antimicrobial wound dressing comprising a wound dressing substrate comprising at least one species of Formula (I), wherein X is a halogen, and wherein the species is complexed with the wound dressing substrate via at least one cationic group of the substrate.

The species may be complexed with the wound dressing substrate via a counterion that is chemically associated with the wound dressing substrate.

In some preferred embodiments, there is provided an antimicrobial wound dressing comprising a wound dressing substrate comprising at least one species of Formula (I), wherein X is a halogen, and wherein the species is complexed with the wound dressing substrate via a counterion that is chemically associated with the wound dressing substrate.

The counterion may be as described above. The wound dressing substrate may be associated with the counterion by ionic and/or electrostatic interactions.

In some embodiments, no greater than 95% of the total number of species of Formula (I) are complexed with the substrate, or no greater than 90, 80, 70, 60, 50, 40, 30, 20, or no greater than 10% of the total number of species are complexed with the substrate.

In some embodiments, the species of Formula (I) is complexed with a non-polymeric counterion and with the wound dressing substrate.

In some preferred embodiments, there is provided an antimicrobial wound dressing comprising a wound dressing substrate comprising at least one species of Formula (I), wherein X is a halogen, and wherein the species is complexed with a non-polymeric counterion and with the wound dressing substrate.

The species of Formula (I) may be complexed with a non-polymeric counterion and with the wound dressing substrate, and the species may not be complexed with any other chemical entity. In some embodiments, a greater number of species are complexed with a non-polymeric counterion than are complexed with the substrate. In other embodiments, a greater number of species are complexed with the substrate than are complexed with a non-polymeric counterion. The ratio of the number of species complexed with a non-polymeric counterion to the number of species complexed with the substrate may be between 1:10 and 10:1. The ratio of the number of species complexed with a non-polymeric counterion to the number of species complexed with the substrate may be between 1:10 and 1:1, or between 1:10 and 1:4, or between 1:10 and 1:8. In some embodiments, the ratio of the number of species complexed with a non-polymeric counterion to the number of species complexed with the substrate may be between 10:1 and 1:1, or between 10:1 and 4:1, or between 10:1 and 8:1.

Such wound dressings benefit from excellent active molecular iodine loadings-minimal additional supporting compounds are required to stably retain the active molecular iodine in the samples, which allows for greatly improved active iodine loadings. Despite the lack of additional supporting compounds, the dressings display optimal active iodine holding and release properties. In dry, out-of-wound environments molecular iodine release is minimal; whereas in moist, wound-based environments, controlled release is initiated to provide strong antimicrobial activity.

In some embodiments, the wound dressing substrate is formed from at least one polymer. At least one polymer may preferably comprise a fibrous polymer. By “fibrous polymer”, it is meant that the polymer is present in a fibrous physical form. At least 25 wt % of the wound dressing substrate may be polymeric, or at least 50, 75, 90, 95, 96, 97, 98, or at least 99 wt % of the wound dressing substrate may be polymeric. In some embodiments, the wound dressing substrate is entirely polymeric. In preferred embodiments, the wound dressing substrate is formed from at least one water-swellable polymer. The wound dressing substrate may be formed from at least one water-absorbing swellable polymer. The wound dressing may be formed from at least one water-soluble polymer.

Use of such wound dressing substrate polymers allows for excellent molecular iodine retention and release properties. In dry, out-of-wound environments molecular iodine release is minimal; whereas in moist, wound-based environments, the substrate polymers are able to expand/swell and allow controlled release of molecular iodine to provide strong antimicrobial activity.

In some embodiments, the wound dressing substrate is formed from at least one polysaccharide or polysaccharide-based material. At least one polysaccharide may preferably comprise a water-swellable polysaccharide. At least one polysaccharide or polysaccharide-based material may be independently chosen from: a carboxyalkylcellulose (preferably carboxymethylcellulose), alginate, a cellulose alkylsulfonate (which may comprise cellulose ethylsulfonate), chitosan, chitin, pectin, hydroxypropyl cellulose, hydroxypropyl methylcellulose, starch (which may comprise konjac), a saccharide-uronic acid co-oligomer or copolymer (which may comprises gellan gum and/or psyllium), and combinations and/or derivatives thereof. At least one polysaccharide or polysaccharide-based material may be independently selected from the group consisting of: a carboxyalkylcellulose (preferably carboxymethylcellulose), alginate, a cellulose alkylsulfonate (which may comprise cellulose ethylsulfonate), chitosan, chitin, pectin, hydroxypropyl cellulose, hydroxypropyl methylcellulose, starch (which may comprise konjac), a saccharide-uronic acid co-oligomer or copolymer (which may comprises gellan gum and/or psyllium), and combinations and/or derivatives thereof.

In some embodiments, the wound dressing substrate is formed from at least one vinyl polymer, which may comprise a water-swellable vinyl polymer. In some embodiments, the wound dressing substrate is formed from at least one vinyl polymer independently chosen from: a poly(vinyl alkanoate) (such as poly(vinyl acetate)), a polyacrylate, a polyalkacrylate, poly(vinyl alcohol), and combinations and/or derivatives thereof. In some embodiments, the wound dressing substrate is formed from at least one vinyl polymer independently selected from the group consisting of: a poly(vinyl alkanoate) (such as poly(vinyl acetate)), a polyacrylate, a polyalkacrylate, poly(vinyl alcohol), and combinations and/or derivatives thereof.

In some embodiments, the wound dressing substrate is formed from at least one ionic polymer. At least one ionic polymer may be independently chosen from: an anionic polymer, a cationic polymer, an amphoteric polymer, and combinations thereof. At least one ionic polymer may be independently selected from the group consisting of: an anionic polymer, a cationic polymer, an amphoteric polymer, and combinations thereof.

In some embodiments, the wound dressing substrate is formed from at least one ionic polymer that is independently selected from: an anionic polymer, an amphoteric polymer, and combinations thereof.

In some preferred embodiments, the wound dressing substrate is formed from at least one anionic polymer. At least one anionic polymer may comprise a water-swellable polymer. At least one anionic polymer may comprise a polysaccharide and/or vinyl polymer, which may preferably be water-swellable. At least one anionic polymer may be independently chosen from: a carboxyalkylcellulose (preferably carboxymethylcellulose), alginate, a poly(vinyl alkanoate) (such as poly(vinyl acetate)), a cellulose alkylsulfonate (which may comprise cellulose ethylsulfonate), a polyacrylate, a polyalkacrylate, and combinations and/or derivatives thereof. At least one anionic polymer may be independently selected from the group consisting of: a carboxyalkylcellulose (preferably carboxymethylcellulose), alginate, a poly(vinyl alkanoate) (such as poly(vinyl acetate)), a cellulose alkylsulfonate (which may comprise cellulose ethylsulfonate), a polyacrylate, a polyalkacrylate, and combinations and/or derivatives thereof.

At least one anionic polymer may comprise at least one polymer having at least one carboxyl moiety, preferably in the form of a carboxylate.

In some embodiments, the wound dressing substrate is formed from at least one amphoteric polymer, which may comprise at least one amphoteric water-swellable polymer. At least one amphoteric polymer may comprise a polysaccharide and/or vinyl polymer. At least one amphoteric polymer may be independently chosen from: chitosan, chitin, gelatin, collagen, and combinations thereof. At least one amphoteric polymer may be independently selected from the group consisting of: chitosan, chitin, gelatin, collagen, and combinations thereof.

The amphoteric polymer may be amphoteric per se or have amphoteric properties.

In some embodiments, the wound dressing substrate is formed from at least one nonionic polymer, which may comprise at least one nonionic water-swellable polymer. At least one nonionic polymer may comprise a polysaccharide and/or vinyl polymer. At least one nonionic polymer may be independently chosen from: poly(vinyl alcohol), pectin, and combinations thereof. At least one nonionic polymer may be independently selected from the group consisting of: poly(vinyl alcohol), pectin, and combinations thereof.

In some embodiments, the wound dressing substrate is formed from at least one cellulosic polymer. Preferably, the cellulosic polymer is a water-swellable polymer. The cellulosic polymer may be a cellulosic anionic polymer.

At least one cellulosic polymer may preferably comprise a carboxyalkylcellulose or a derivative thereof. At least one cellulosic polymer may comprise a C1-C10 carboxyalkylcellulose, C1-C5, or C1-C3 carboxyalkylcellulose. At least one cellulosic polymer may be independently chosen from: carboxymethylcellulose, carboxyethylcellulose, and combinations thereof. At least one cellulosic polymer may be independently selected from the group consisting of: carboxymethylcellulose, carboxyethylcellulose, and combinations thereof. At least one cellulosic polymer may preferably comprise carboxymethylcellulose (CMC) or a derivative thereof. At least one cellulosic polymer may be cross-linked.

Carboxymethylcellulose refers to cellulose containing pendant carboxymethyl groups bound to one or more of the hydroxyl groups of the glucose monomers that make up the cellulose backbone. In some embodiments, the wound dressing substrate is formed from at least one carboxymethylcellulose polymer having a degree of substitution of between 0.05-0.5, preferably between 0.15-0.4, or more preferably between 0.25-0.35; which is intended to mean that from 0.05-0.5 carboxymethyl groups are provided per glucose unit.

In some preferred embodiments, there is provided a wound dressing substrate comprising at least one species of Formula (I), wherein X is a halogen, and wherein the species is complexed with a non-polymeric counterion and/or complexed with the wound dressing substrate, wherein the wound dressing substrate is formed from at least one carboxymethylcellulose polymer having a degree of substitution of between 0.05-0.5.

In some embodiments, the wound dressing substrate is formed from at least one carboxymethylcellulose polymer having a degree of substitution of greater than 0.5, such as between 0.5-0.8.

In some preferred embodiments, there is provided a wound dressing substrate comprising at least one species of Formula (I), wherein X is a halogen, and wherein the species is complexed with a non-polymeric counterion and/or complexed with the wound dressing substrate, wherein the wound dressing substrate is formed from at least one carboxymethylcellulose polymer having a degree of substitution of greater than 0.5.

In embodiments in which the wound dressing substrate is formed from at least one carboxymethylcellulose polymer having a degree of substitution of greater than 0.5, the carboxymethylcellulose polymer may preferably be cross-linked. A carboxymethyl group will be understood to be a —CH₂COOH group, wherein the carboxymethyl group is bound to the cellulose backbone via a pendant hydroxyl group to form an ether bond i.e. —OCH₂COOH.

In some embodiments, the wound dressing substrate may be formed for at least one material having a homogenous structure and/or a heterogenous structure. In some embodiments, the wound dressing substrate is formed from at least one material type independently chosen from: woven, nonwoven, gauze, fibrous, sheet, film, knitted, and combinations thereof. In some embodiments, the wound dressing substrate is formed from at least one material type independently selected from the group consisting of: woven, nonwoven, gauze, fibrous, sheet, film, knitted, and combinations thereof. A nonwoven material may comprise a felt (which may be a needle-punched felt) or a hydroentangled sheet. The wound dressing substrate may be stitched. The wound dressing substrate may be formed from at least one material independently chosen from: a fibre-based material, a yarn-based material, a filament-based material, a multifilament-based material, and combinations thereof. The wound dressing substrate may be formed from at least one material independently selected from the group consisting of: a fibre-based material, a yarn-based material, a filament-based material, a multifilament-based material, and combinations thereof. In some embodiments, the wound dressing substrate may be formed from at least one fibrous nonwoven material, which may comprise a needle punched felt or hydroentangled sheet. The wound dressing may be formed from at least one water-swellable fibrous material, which may comprise a water-swellable fibrous nonwoven material.

In preferred embodiments, the wound dressing substrate is formed from at least one polymer material. The polymer material type may be as described in statements above. At least one polymer may preferably be as described in statements of invention above. In preferred embodiments, at least one polymer is a water-swellable polymer, preferably as described above. At least one polymer may comprise a cellulosic polymer, which may preferably comprise CMC or a derivative thereof. At least one polymer material may preferably comprise a fibrous polymer material, preferably a fibrous water-swellable polymer material. The wound dressing substrate may be formed of fibres containing at least 60 wt. % of polymer, preferably water-swellable polymer, or at least 65, 70, 75, 80, 85, 90, or at least 95, or 100 wt. % of polymer, preferably water-swellable polymer. The fibres may preferably comprise a water-swellable polymer, as described above. The water-swellable polymer may be or comprise a cellulosic polymer, preferably CMC or a derivative thereof. The wound dressing substrate may be formed of fibres containing at least 60 wt. % of a cellulosic polymer, preferably CMC, or at least 65, 70, 75, 80, 85, 90, 95, 96, 97, 98, or at least 99 wt. %, or 100 wt. % of a cellulosic polymer, preferably CMC.

In some embodiments, the wound dressing substrate has a total thickness of at least 0.25 mm, or at least 0.5, 0.75, 1, 2, 3, 4, 5, 6, 7, 8, 9, or at least 10 mm. In some embodiments, the wound dressing substrate may have a total thickness of no greater than 30 mm, or no greater than 25, 20, 15, 10, 5, 4, 3, 2, or no greater than 1 mm.

In some embodiments, the wound dressing substrate comprises a singular piece, which may be a layer (single layer wound dressing). The piece may be a wound contact layer. The wound contact layer may comprise a first surface for contacting a wound, in use, and an opposing second surface. At least one species of Formula (I) may be present in and/or on the wound contact layer. The wound contact layer may be formed from a water-swellable polymer, preferably as described above. The wound contact layer may be formed from a cellulosic polymer, preferably cellulosic polymer fibres. The wound contact layer may be formed of carboxymethylcellulose fibres. In some embodiments, the singular piece wound dressing may be used in conjunction with a secondary wound dressing. The secondary dressing may include a semi-permeable layer.

Alternatively, the wound dressing substrate may comprise more than one piece. In some embodiments, the wound dressing substrate has a layered structure. The wound dressing substrate may be a multi-layer substrate. In some embodiments, the wound dressing substrate comprises 2 or at least 2 layers, or 3 or at least 3, 4 or at least 4, or 5 or at least 5 layers.

In some embodiments, the wound dressing substrate comprises a backing layer and a wound contact layer. The wound contact layer may be as described above. The backing layer may have a first surface facing the wound contact layer and an opposing second surface. The wound dressing substrate may comprise a backing layer, wound contact layer, and an adhesive layer for adhering the wound dressing to a wound, in use. The wound contact layer may comprise a first surface for directly contacting a wound, in use, and a second surface facing the backing layer. The wound-site adhesive layer may be perforated and comprise a plurality of perforations. In such embodiments, at least one species of Formula (I) may be present in and/or on the wound contact layer.

The adhesive layer may comprise a border region which surrounds the wound contact layer. The perforations may have a diameter between 1.6 mm and 2.7 mm. In some embodiments, the perforations may preferably comprise a diameter between 2.1 mm and 2.3 mm, more preferably 2.2 mm.

The adhesive layer may be restricted to the border region which surrounds the wound contact layer. In some embodiments, the adhesive layer may partially overlap the wound contact layer. The adhesive layer may adhere to the wound contact layer so as to hold the wound contact layer in a fixed relation to the backing layer and/or the wound site.

In some embodiments, the adhesive layer may extend continuously across the first surface of the wound contact layer. Hence, the contact between the wound contact layer and wound may be through the perforations.

The perforations may be uniformly distributed across the full extent of the adhesive layer. In some embodiments, the perforations in the border region may differ from the perforations in a central region which corresponds to the wound contact layer. The perforations between the central region and border region may differ in size, shape, distribution (i.e. pattern) or number. The perforations in the border region may be uniform. The perforations in the central region may be uniform. The perforations may have a common size. The common size may be a common diameter. Hence, the diameter of the perforations may all be 2.2 mm, for example.

The adhesive layer may comprise a silicone adhesive. The adhesive layer may comprise an adhesive having a density of between 70 g and 150 g per square meter.

The adhesive layer may comprise an inner edge which defines a central window (which may be referred to as a central region) through which the wound contact layer may be exposed.

The wound contact layer may comprise an outer edge and may overlap the adhesive layer around the periphery of the wound contact layer so as to be adhered thereto. The width of the overlap between the inner edge of the adhesive layer and the outer edge of the wound contact layer may vary around the periphery of the wound contact layer.

The overlap may comprise one or more corner region and one or more edge region of the wound contact layer and adhesive layer. The width of the overlap in the corner regions may be greater than the overlap in an adjacent edge region.

The one or more corner region may comprise an inner edge having a first radius of curvature, and the outer edge comprises a second radius of curvature. The first radius of curvature may be greater than the second radius of curvature.

The backing layer may be adhered to the adhesive layer. The wound dressing substrate may further comprise a superabsorbent layer. The superabsorbent layer may be in adhesive-free contact with the backing layer.

The superabsorbent layer may comprise a first surface facing the wound contact layer and a second surface facing the backing layer. The superabsorbent layer first surface may comprise an adhesive layer.

In some embodiments, the wound dressing substrate may comprise: a backing layer; a wound contact layer having a first surface for directly contacting a wound bed and a second surface facing the backing layer, and a superabsorbent layer.

The wound contact layer may comprise gel forming fibres. The superabsorbent layer may be located between the backing layer and the second surface of the wound contact layer.

The wound contact layer may further comprise a foam layer located between the backing layer and the second surface of the wound contact layer. The foam layer may be located between the wound contact layer and the superabsorbent layer.

The superabsorbent layer may comprise fibres, optionally nonwoven fibres. The superabsorbent layer may comprise nonwoven fibres only.

The superabsorbent layer may have a thickness of between 1.5 mm and 2.5 mm, optionally 2 mm.

The superabsorbent layer may comprise a first surface facing the wound contact layer and a second surface facing the backing layer. The superabsorbent layer first surface may comprise an intra-layer adhesive. The intra-layer adhesive may be a continual layer, perforated layer, liquid or scatter coat adhesive for example. The intra-layer adhesive may be a web comprising a continuous or broken layer of adhesive which is inserted in between the respective adjacent layers and activated to bond the layers together. The intra-layer adhesive may be thermally activated for example in which heat and pressure are applied to during a lamination process. The intra-layer adhesive may comprise a web of adhesive strands or dots.

The super-absorbent layer second surface may be configured to be movable relative to the backing layer. The superabsorbent layer may contact the backing layer. The contact between the superabsorbent layer may allow for relative movement, e.g. sliding contact or differential expansive contact.

The backing layer may comprise an adhesive border region which surrounds a non-adhesive central region. The superabsorbent layer may be located fully within the non-adhesive central region such that the superabsorbent layer is not adhered to the backing layer.

The foam layer may comprise a first, wound facing surface and a second, backing layer facing surface. The foam layer first surface may comprise a foam first adhesive layer. The second surface of the foam layer may comprise a foam second adhesive layer. The foam layer and superabsorbent layer may be adhered together.

The foam layer may comprise a polyurethane foam. The foam layer may comprise an aliphatic foam or a methylene diphenyl disoocyanate foam.

The foam layer may have a thickness of between 1.3 mm and 3.2 mm, optionally 2.5 mm.

The wound contact layer may have a thickness between 1 mm and 1.5 mm. The wound contact layer may have a base weight of between 70 gsm and 150 gsm, optionally 70 gsm.

The backing layer may comprise a polyurethane material. The backing layer may have a thickness of 30 microns.

In some embodiments, the wound dressing substrate comprises: a wound contact layer; a backing layer; an intermediate layer located between the backing layer and wound contact layer. The intermediate layer may comprise a first surface facing the wound contact layer and a second surface facing the backing layer.

The first surface of the intermediate layer may be bonded directly or indirectly to the wound contact layer. The second surface may be unbonded to the backing layer.

The dressing substrate may further comprise a wound-site adhesive layer provided on a peripheral region of the backing layer for adhering the backing layer to a wound site.

The intermediate layer may be adhered directly or indirectly to the wound contact layer.

The wound dressing substrate may further comprise an adhesive to provide the adherence of the intermediate layer to the wound contact layer. The adhesive may be referred to as an intra-layer adhesive or a binder layer. The adhesive may comprise a scatter coat adhesive.

The intermediate layer may comprise a superabsorbent layer.

The wound dressing substrate may further comprise a foam layer located between the wound contact layer and backing layer. The foam layer may be located between the wound contact layer and the first surface of the superabsorbent layer. The foam layer may be adhered to the wound contact layer.

The wound-site adhesive layer may comprise a border region of adhesive which surrounds a central region in which the wound contact layer is located. The border region may partially cover the wound contact layer. The adhesive layer may comprise an inner edge defining a central window through which the wound contact layer is exposed.

In some embodiments, the wound dressing substrate comprises a membrane provided over at least one outer surface of the substrate. The membrane may have an open structure, preferably to allow molecular iodine to be released from the substrate. The membrane may be independently selected from: a gauze, a mesh, a layer, a woven material, and a nonwoven material. In some embodiments, the membrane encapsulates part of the substrate which comprises at least one species of Formula (I). The membrane may encapsulate said part partially or fully. The membrane may comprise a layer that is on said part. The membrane may be perforated. The membrane may comprise a wound contacting layer, which may be perforated. In some embodiments, the wound contacting layer membrane comprises an adhesive to adhere to a patient's skin, in use. In some embodiments, the membrane comprises at least two layers, wherein one layer is a perforated wound-contacting layer, and a second layer is a semi-permeable layer. In some embodiments, the membrane is present on a wound contacting surface of the wound contact layer.

In some embodiments, the wound dressing substrate is part of a negative pressure wound therapy (NPWT) dressing.

In some embodiments, the wound dressing substrate comprises an absorbent layer. The wound dressing substrate may be a multilayer substrate comprising an absorbent layer. The absorbent layer may be a wound contact layer or may not be a wound contact layer. In some embodiments, the absorbent layer is not a wound contact layer and is an intermediate layer between an outer wound contact layer and a backing layer. In all such embodiments, at least one species of Formula (I) may be present in and/or on the absorbent layer.

At least one species of Formula (I) may be retained within the wound dressing substrate. At least one species may be retained within the structure of the wound dressing substrate, preferably within the three-dimensional structure. In some embodiments, the wound dressing substrate is formed from at least one polymer, preferably as described in statements of invention above, and at least one species of Formula (I) is held within the polymer structure, preferably within the three-dimensional structure of the polymer. In some embodiments, the wound dressing substrate comprises a polymer matrix, and at least one species may be retained within the polymer matrix. In some embodiments, the wound dressing substrate comprises entangled fibres, preferably entangled polymer fibres. At least one species of Formula (I) may be retained by the entangled fibres. At least one species of Formula (I) may be retained between entangled fibres. In embodiments wherein the wound dressing substrate is a multi-layer substrate, at least one species of Formula (I) may be present in and/or on a wound contact layer of the substrate. At least one species of Formula (I) may be present in and/or on an absorbent layer of the substrate, which may be a wound contact layer or may not be a wound contact layer. At least one species of Formula (I) may be present in and/or on an intermediate layer of a multi-layer substrate, which may be an absorbent layer.

In some embodiments, the wound dressing substrate comprises an open structure. The wound dressing substrate may comprise an open internal and/or external structure. The wound dressing substrate may comprise at least one interstitial region. At least one species of Formula (I) may be retained within at least one interstitial region of the wound dressing substrate. At least one interstitial region may comprise a three-dimensional space within the structure of the substrate, preferably within the polymer structure of the substrate, for example as spaces between fibres. In some embodiments, at least one species of Formula (I) is adsorbed to the wound dressing substrate, preferably to a surface thereof. At least one species of Formula (I) may be physisorbed and/or chemisorbed to the wound dressing substrate.

In some embodiments, at least one species of Formula (I) may be retained within the wound dressing substrate (which may be as described above) and/or adsorbed to the wound dressing substrate by one or more of: intermolecular forces, electrostatic interactions, and ionic interactions between the species and the substrate. In some embodiments, at least one species may be retained and/or adsorbed by chemical association with the substrate, which may involve chemical association with one or more of: a material, yarn, fibre, and filament thereof.

In embodiments in which at least one species of Formula (I) forms a complex with the substrate, at least one species of Formula (I) may be retained within the wound dressing substrate and/or adsorbed to the substrate by the complex formed between the species and the wound dressing substrate. The species may only be retained and/or adsorbed by the complex formed between the species and the wound dressing substrate. Alternatively, the species may be retained and/or adsorbed by the complex formed and by electrostatic interactions between the species and substrate.

In some embodiments, X (as in Formula (I)) is a halogen. X may be a halogen that is independently chosen from: fluorine, chlorine, bromine, and iodine. X may be a halogen that is independently selected from the group consisting of: fluorine, chlorine, bromine, and iodine. In some embodiments, X is independently chosen from: chlorine and iodine. In some embodiments, X is independently selected from the group consisting of: chlorine and iodine. In some embodiments, X is iodine and at least one species is triiodide.

In some embodiments, the dressing comprises 2 or at least 2 different species of Formula (I), or 3 or at least 3, or 4 different species of Formula (I). In some embodiments, the dressing comprises no greater than 3, or no greater than 2 different species of Formula (I). In some embodiments, the dressing comprises a single species of Formula (I). At least one species is preferably I—I—I. In some embodiments one species may be I—I—I and at least one further species may be chosen from I—I—Cl, I—I—Br and I—I—F. In some embodiments one species may be I—I—I and at least one further species may be selected from the group consisting of I—I—Cl, I—I—Br and I—I—F.

In some embodiments, the species of Formula (I) is present in a total amount of at least 0.025 wt. % of the wound dressing substrate, or at least 0.05, 0.1, 0.2, 0.3, 0.4, or at least 0.5, 0.6, 0.7, or at least 0.8 wt % of the wound dressing substrate. In some embodiments, the species of Formula (I) is present in a total amount of no greater than 30 wt. % of the wound dressing substrate, or no greater than 25, 20, 19, 18, 17, 16, or no greater than 15 wt % of the wound dressing substrate. The species of Formula (I) may be present in a total amount of between 0.025-25 wt % of the wound dressing substrate, or between 0.05-20, 0.25-20, or between 0.5-18 wt % of the wound dressing substrate. The species of Formula (I) may be present in a total amount of between 0.1-5 wt % of the wound dressing substrate, or between 0.25-2.5, or between 0.5-2 wt % of the wound dressing substrate. The species of Formula (I) may be present in a total amount of between 5-25 wt % of the wound dressing substrate, 8-20, or between 10-18 wt % of the wound dressing substrate.

In some embodiments, the wound dressing substrate further comprises at least one halide, preferably free halide. At least one halide may be the same as X in Formula (I). In some embodiments, at least one halide is independently chosen from: fluoride, chloride, bromide, and iodide. In some embodiments, at least one halide is independently selected from the group consisting of: fluoride, chloride, bromide, and iodide. At least one halide may be independently chosen from: chloride and iodide. At least one halide may be independently selected from the group consisting of: chloride and iodide. At least one halide may be in equilibrium between being a free halide and being part of the species of Formula (I). The equilibrium may be a dynamic equilibrium.

The wound dressing substrate may comprise 2 or at least 2 different halides, or 3 or at least 3 different halides, or 4 different halides. The wound dressing substrate may comprise no greater than 3 different halides, or no greater than 2 different halides. In some embodiments, the wound dressing substrate comprises a single halide.

The halide may be present in a total amount of at least 0.05 wt % of the wound dressing substrate, or at least 0.1, 0.12, 0.14, 0.16, 0.18, 0.2, 0.21, 0.22, 0.23, 0.24, or at least 0.25 wt %. The halide may be present in a total amount of no greater than 10 wt. % of the wound dressing substrate, or no greater than 9, 8, 7, 6 or no greater than 5 wt % of the wound dressing substrate. The halide may be present in a total amount of between 0.05-10 wt % of the wound dressing substrate, or between 0.1-8, 0.15-7, 0.2-6, 0.25-6, 0.25-5, or between 0.25-4 wt % of the wound dressing substrate.

The ratio of the total amount of free halide to the total amount of the species of Formula (I) in the wound dressing may be between 1:10 and 10:1, or between 1:10 and 5:1, or between 1:10 and 1:1, or between 1:10 and 1:5.

In some embodiments, the wound dressing substrate further comprises non-complexed molecular iodine. The non-complexed molecular iodine may be present in an amount of at least 0.0001 wt. % of the wound dressing substrate, or at least 0.0002, 0.0003, 0.0004, 0.0005, 0.0006, 0.0007, 0.0008, 0.0009, or at least 0.001 wt. % of the wound dressing substrate. The non-complexed molecular iodine may be present in an amount of no greater than 0.1 wt. % of the wound dressing substrate, or no greater than 0.9, 0.8, 0.7, 0.6, 0.5, 0.4, 0.3, 0.2, or no greater than 0.1 wt. % of the wound dressing substrate.

In some embodiments, at least one species of Formula (I) is present at and/or on a surface of the wound dressing substrate. The surface is preferably an outer surface. The surface may be a wound-contacting surface and/or a non-wound contacting surface. In some embodiments, the surface may be an inner surface, which may comprise a surface of an internal layer in a multi-layer wound dressing.

In some embodiments, the species of Formula (I) is present at and/or on at least 0.5% of the total area of the surface of the wound dressing substrate, or at least 1, 2, 3, 4, 5, or at least 10, 15, 20, 25, 30, 35, 40, 45, 50, 55, 60, 65, 70, 75, 80, 85, 90, 95, 96, 97, 98, or at least 99% of the total area of the surface of the wound dressing substrate. In some embodiments, the species of Formula (I) is present at and/or on no greater than 95% of the total area of the surface of the wound dressing substrate, or no greater than 90, 85, or no greater than 80% of the total area of the surface of the wound dressing substrate.

In some embodiments, the species of Formula (I) is present in a total amount of between 0.025-25 wt % of the wound dressing substrate, or between 0.05-20, 0.25-20, or between 0.5-18 wt % of the wound dressing substrate; and the species of Formula (I) is present at and/or on at least 25% of the total area of the surface of the wound dressing substrate, or at least 50%, or at least 75% of the total area of the surface of the wound dressing substrate.

In some embodiments, the species of Formula (I) is present at and/or on the surface of the wound dressing substrate at a surface density of at least 0.25 mg/cm², or at least 0.5, 0.75, 1, 1.25, 1.5, 1.75, 2, 2.25, 2.5, 2.75, or at least 3 mg/cm². The species of Formula (I) may be present at and/or on the surface of the wound dressing substrate at a surface density of no greater than 10 mg/cm², or no greater than 9, 8, 7, 6, 5, or no greater than 4 mg/cm². The species of Formula (I) may be present at and/or on the surface of the wound dressing substrate at a surface density of between 0.25-8 mg/cm², or between 0.5-6 mg/cm², or between 1-5 mg/cm².

In some preferred embodiments, there is provided a wound dressing substrate comprising at least one species of Formula (I), wherein X is a halogen, and wherein the species is complexed with a non-polymeric counterion and/or complexed with the wound dressing substrate, wherein the species is present at and/or on a surface of the wound dressing substrate at a surface density of between 0.25-8 mg/cm².

The species of Formula (I) may be present at and/or on the surface of the wound dressing substrate at a surface density of between 0.25-8 mg/cm², or between 0.5-⁶, or between 1-5 mg/cm²; and the species of Formula (I) is present at and/or on at least 25% of the total area of the surface of the wound dressing substrate, or at least 50%, or at least 75% of the total area of the surface of the wound dressing substrate.

In some embodiments, the species of Formula (I) is present at and/or on the surface of the wound dressing substrate at a uniform surface density. In such embodiments, the wound dressing substrate may be formed from a film material type. In other embodiments, the species of Formula (I) may be present at and/or on the surface at a non-uniform surface density. In such embodiments, the wound dressing substrate may be formed from a material having a heterogenous structure, which may be a nonwoven material. In some embodiments, the surface is a wound-contacting surface having a central portion and sides. The species may be present at a higher concentration at the central portion of the surface than at the sides. The species may alternatively be present at a higher concentration at the sides than at the central portion.

In some embodiments, at least 2.5% of the total amount of the species of Formula (I) is present at and/or on the surface of the wound dressing substrate, or at least 5, 7.5, 10, 12.5, 15, 17.5, 20, 25, 30, 35, 40, 45, 50, 55, 60, 65, 70, 75, 80, 85, 90, 95, 96, 97, 98, or at least 99% of the total amount of the species is present at and/or on the surface of the substrate. In some embodiments, 100% of the total amount of the species is present at and/or on the surface of the substrate. In some embodiments, no greater than 95% of the total amount of the species is present at and/or on the surface of the substrate, or no greater than 90, 85, 80, 75, 70, 65, 60, 55, 50, 45, 40, 35, 30, 25, 20, 15, 10, or no greater than 5% of the total amount of the species is present at and/or on the surface of the substrate. In some embodiments, none of the species is present at and/or on the surface, and substantially all of the species may be located within the wound dressing substrate.

In some embodiments, the species of Formula (I) is present in a total amount of between 0.025-25 wt % of the wound dressing substrate, or between 0.05-20, 0.25-20, or between 0.5-18 wt % of the wound dressing substrate; and at least 25% of the total amount of the species of Formula (I) is present at and/or on the surface of the wound dressing substrate, or at least 50%, or at least 75% of the total amount of the species of Formula (I) is present at and/or on the surface of the wound dressing substrate.

In some embodiments, at least 25% of the total amount of the species of Formula (I) is present at and/or on the surface of the wound dressing substrate, or at least 50%, or at least 75% of the total amount of the species of Formula (I) is present at and/or on the surface of the wound dressing substrate; and the species of Formula (I) is present at and/or on the surface of the wound dressing substrate at a surface density of between 0.25-8 mg/cm², or between 0.5-6, or between 1-5 mg/cm².

In some embodiments, at least 25% of the total amount of the species of Formula (I) is present at and/or on the surface of the wound dressing substrate, or at least 50%, or at least 75% of the total amount of the species of Formula (I) is present at and/or on the surface of the wound dressing substrate; and the species of Formula (I) is present at and/or on at least 25% of the total area of the surface of the wound dressing substrate, or at least 50%, or at least 75% of the total area of the surface of the wound dressing substrate.

In some embodiments, at least 25% of the total amount of the species of Formula (I) is present at and/or on the surface of the wound dressing substrate, or at least 50%, or at least 75% of the total amount of the species of Formula (I) is present at and/or on the surface of the wound dressing substrate; the species of Formula (I) is present at and/or on at least 25% of the total area of the surface of the wound dressing substrate, or at least 50%, or at least 75% of the total area of the surface of the wound dressing substrate; and the species of Formula (I) is present at and/or on the surface of the wound dressing substrate at a surface density of between 0.25-8 mg/cm², or between 0.5-6, or between 1-5 mg/cm².

In some embodiments, at least one species of Formula (I) is located within the wound dressing substrate and at a depth beneath the outer surface of the substrate of at least 0.025 mm, or at least 0.05, 0.075, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, or at least 50 mm. The species of Formula (I) may be located at a depth of beneath the outer surface of the substrate of no greater than 100 mm, or no greater than 90, 80, 70, 60, 50, 40, 30, 20, 10, 9, 8, 7, 6, 5, 4, 3, 2, 1, 0.5, or no greater than 0.05 mm.

In some embodiments, the wound dressing has a releasable iodine content of at least 0.025 wt. % of the wound dressing substrate, or at least 0.05, 0.1, 0.2, 0.3, 0.4, or at least 0.5, 0.6, 0.7, or at least 0.8 wt % of the wound dressing substrate. The wound dressing may have a releasable iodine content of no greater than 30 wt. % of the wound dressing substrate, or no greater than 25, 20, 19, 18, 17, 16, or no greater than 15 wt % of the wound dressing substrate. In some embodiments, the antimicrobial wound dressing has a releasable iodine content of between 0.025-25 wt. % of the wound dressing substrate, or between 0.05-20, 0.25-20, or between 0.5-18 wt. % of the wound dressing substrate.

In some preferred embodiments, there is provided a wound dressing substrate comprising at least one species of Formula (I), wherein X is a halogen, and wherein the species is complexed with a non-polymeric counterion and/or complexed with the wound dressing substrate, wherein the antimicrobial wound dressing has a releasable iodine content of between 0.5-18 wt. % of the wound dressing substrate.

In some embodiments, the wound dressing may comprise at least one moistening liquid, which may be independently chosen from: water, an aqueous solution, an organic liquid, an organic solution, and combinations thereof. In some embodiments, the wound dressing may comprise at least one moistening liquid, which may be independently selected from the group consisting of: water, an aqueous solution, an organic liquid, an organic solution, and combinations thereof. In some embodiments, the wound dressing has a total moisture content of at least 0.5 wt. % of the dressing, or at least 1, 2, 3, 4, 5, 6, 7, 8, 9, or at least 10 wt. % of the dressing. The wound dressing may have a total moisture content of no greater than 60 wt. % of the dressing, or no greater than 50, 40, 30, 20, 10, 9, 8, 7, 6, 5, 4, 3, 2, 1, or of no greater than 0.5 wt. % of the dressing. In some embodiments, the dressing may contain substantially no moisture and may be fully dry. In some embodiments, the wound dressing has a total moisture content of between 5-60 wt. %, 10-60 wt. %, 15-55 wt. %, 20-50 wt. %, 25-50 wt. %, 30-50 wt. %, 30-45 wt. %, or of between 35-40 wt. %. In other embodiments, the wound dressing has a total moisture content of between 0-60 wt. %, or between 0-50, 0-40, 0-30, 0-20, 0-10, or between 0-5 wt. % of the dressing.

According to a second aspect of the invention, there is provided a method of preparing an antimicrobial wound dressing comprising the steps of:
(a) Providing a wound dressing substrate;
(b) Dissolving molecular iodine and at least one halide-containing species in a solvent to provide a solution; and
(c) Treating the wound dressing substrate with the solution to provide the antimicrobial wound dressing.

The use of molecular iodine and a halide-containing species in step (b) allows for a wound dressing with excellent active molecular iodine uptake and binding. The molecular iodine is believed to bind with the halide from the halide-containing species to form a complex ion which is retained in the dressing with minimal to no premature iodine release pre-exposure to a wound. On wound exposure, controlled molecular iodine release is initiated to deliver strong antimicrobial activity.

In some embodiments, the solvent in step (b) comprises at least one organic solvent, preferably at least one polar organic solvent. At least one polar organic solvent may be as described for the third aspect of the invention below.

In some embodiments, the solvent in step (b) comprises water and a water-miscible polar organic solvent.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solvent comprises water and a water miscible polar organic solvent; and
(c) Treating the wound dressing substrate with the solution to provide the antimicrobial wound dressing.

The amount by volume of the water-miscible polar organic solvent may be greater than or equal to the amount by volume of water in the solvent.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solvent comprises water and a water miscible polar organic solvent, wherein the amount by volume of the water-miscible polar organic solvent is greater than or equal to the amount by volume of water in the solvent; and
(c) Treating the wound dressing substrate with the solution to provide the antimicrobial wound dressing.

The solvent in step (b) may comprise water and a water-miscible polar organic solvent in a water:polar organic solvent ratio of between 5:95 and 50:50 vol.:vol. The solvent in step (b) may be as described for the third aspect of the invention below.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solvent comprises water and a water miscible polar organic solvent in a water:polar organic solvent ratio of between 5:95 and 50:50 vol.:vol.; and
(c) Treating the wound dressing substrate with the solution to provide the antimicrobial wound dressing.

In other embodiments, the solvent in step (b) may comprise substantially no water. The solvent in step (b) may only comprise an organic solvent, which may be as described for the third aspect of the invention below.

According to a third aspect of the invention, there is provided a method of preparing an antimicrobial wound dressing comprising the steps of:
(a) Providing a wound dressing substrate;
(b) Dissolving molecular iodine in a solvent comprising water and a water-miscible polar organic solvent in a water:polar organic solvent ratio of between 5:95 and 50:50 vol.:vol. to provide a solution; and
(c) Treating the wound dressing substrate with the solution to provide the antimicrobial wound dressing.

Use of such a solvent mixture in step (b) of the method provides a wound dressing with good active iodine binding and an even and undisturbed distribution of iodine throughout the dressing.

In some embodiments, step (b) comprises dissolving molecular iodine and at least one halide-containing species in the solvent, preferably as described for the second aspect of the invention above.

The following statements apply to the second and third aspects of the invention.

The antimicrobial wound dressing substrates of the second and third aspects of the invention are preferably the antimicrobial wound dressing substrate of the first aspect of the invention. Statements of invention above relating to the antimicrobial wound dressing substrates of the first aspect of the invention or to any components thereof may also be applied to the second and third aspects of the invention. Other statements of invention relating to the first aspect of the invention may also be applied mutatis mutandis to the second and third aspects of the invention.

In some preferred embodiments, the wound dressing substrate comprises fibrous CMC. Step (a) may comprise forming a wound dressing substrate comprising fibrous CMC by reacting cellulose fibres with a carboxymethylating agent to provide fibrous CMC. Cellulose fibres may be provided as continuous-filament cellulose. Cellulose fibres may be natural or synthetic. Cellulose fibres may be derived from wood pulp (e.g. Eucalyptus, Oak, Birch wood, soft wood). Reacting the cellulose fibres with a carboxymethylating agent may comprise contacting the cellulose fibres with a carboxymethylating agent, and preferably a base. The carboxymethylating agent may comprise a haloacetic acid salt, preferably chloroacetic acid salt, which may comprise a monochloroacetate, such as sodium monochloroacetate. The base may comprise an alkali metal hydroxide, such as sodium hydroxide.

The cellulose fibres may be contacted with the base and the carboxymethylating agent concurrently, or sequentially. In some embodiments, the carboxymethylating agent and base are provided in a carboxymethylating solution and the cellulose fibres are submerged in the carboxymethylating solution. The carboxymethylating solution comprises the carboxymethylating agent, and optionally the base. The carboxymethylating agent may comprise 2-8 wt. % carboxymethylating agent, or 3-7, or 4-6 wt. % carboxymethylating agent. The carboxymethylating solution may comprise between 0.5-8 wt % base, or between 1-7, or between 2-4 wt. % base. In some embodiments, the carboxymethylating solution comprises a water-miscible organic solvent, which may be as described in statements below relating to the solvent used in step (b) of the method. In some embodiments, at least one water-miscible organic solvent is independently chosen from: C1-C4 alcohols, C3-C5 ketones, and combinations thereof. In some embodiments, at least one water-miscible organic solvent is independently selected from the group consisting of: C1-C4 alcohols, C3-C5 ketones, and combinations thereof. In some embodiments, the carboxymethylating solution comprises greater than 70 wt. % of a water-miscible organic solvent, which may be independently chosen from: C1-C4 alcohols, C3-C5 ketones, and combinations thereof. In some embodiments, the carboxymethylating solution comprises greater than 70 wt. % of a water-miscible organic solvent, which may be independently selected from the group consisting of: C1-C4 alcohols, C3-C5 ketones, and combinations thereof. In some embodiments, the carboxymethylating solution comprises the water-miscible organic solvent in an amount of from 70-85 wt %, optionally 75-80 wt %, or between 76-78 wt %. In some embodiments, the carboxymethylating solution further comprises water, which may be present in an amount of between 5-20 wt %, optionally 7-18 wt %, or between 9-16 wt %, or between 11-14 wt %. The carboxymethylating solution may comprise a solvent as used in step (b) of the method. Statements of invention relating to the solvent of step (b) may also be applied to the solvent used in the carboxymethylating solution.

In some embodiments, the step of reacting the cellulose fibres with a carboxymethylating agent comprises submerging the cellulose fibres in a carboxymethylating solution as described above for between 0.5-3.5 hours, or between 1-3, or between 1.5-2.5 hours. In some embodiments, the carboxymethylating solution is maintained at a temperature of between 55-85° C., or between 60-80, 65-75, or between 68-73° C. In some embodiments, following the step of submerging the cellulose fibres in the carboxymethylating solution, CMC fibres are allowed to cool to a temperature of between 40-60° C., or between 45-55° C., which may be performed over a time period of between 10-20 minutes, or between 14-16 minutes.

In some embodiments, step (a) further comprises the step of washing the wound dressing substrate with a pre-treatment wash solution. The pre-treatment wash solution may be the same as the treatment solvent used in step (b). Statements of invention relating to the solvent used in step (b) may also be applied to the pre-treatment wash solution. Washing the wound dressing substrate with the pre-treatment wash solution may be performed by any suitable wash method, and may include submerging the substrate in the pre-treatment wash solution.

In some embodiments, the polar organic solvent in step (b) comprises at least one solvent independently chosen from: a polar protic organic solvent, a polar aprotic organic solvent, and combinations thereof. In some embodiments, the polar organic solvent in step (b) comprises at least one solvent independently selected from the group consisting of: a polar protic organic solvent, a polar aprotic organic solvent, and combinations thereof. In preferred embodiments, the polar organic solvent is or comprises a water-miscible solvent.

In some embodiments, the polar organic solvent in step (b) comprises at least one polar protic organic solvent, which may preferably be a water-miscible solvent.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solvent comprises water and a water miscible polar protic organic solvent; and
(c) Treating the wound dressing substrate with the solution to provide the antimicrobial wound dressing.

In some preferred embodiments, the method of preparing an antimicrobial wound dressing comprises the steps of:
(a) Providing a wound dressing substrate;
(b) Dissolving molecular iodine in a solvent comprising water and a water-miscible polar organic solvent in a water:polar organic solvent ratio of between 5:95 and 50:50 vol.:vol. to provide a solution, wherein the polar organic solvent comprises a water miscible polar protic organic solvent;
(c) Treating the wound dressing substrate with the solution to provide the antimicrobial wound dressing.

In some embodiments, the polar organic solvent may solely comprise polar protic solvent(s). At least one polar protic organic solvent may be independently chosen from: a carboxylic acid, a phenol, an alcohol, and combinations thereof. At least one polar protic organic solvent may be independently selected from the group consisting of: a carboxylic acid, a phenol, an alcohol, and combinations thereof.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solvent comprises water and a water miscible polar protic organic solvent that is independently chosen from: a carboxylic acid, a phenol, an alcohol, and combinations thereof; and
(c) Treating the wound dressing substrate with the solution to provide the antimicrobial wound dressing.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solvent comprises water and a water miscible polar protic organic solvent that is independently selected from the group consisting of: a carboxylic acid, a phenol, an alcohol, and combinations thereof; and
(c) Treating the wound dressing substrate with the solution to provide the antimicrobial wound dressing.

In some preferred embodiments, the method of preparing an antimicrobial wound dressing comprises the steps of:
(a) Providing a wound dressing substrate;
(b) Dissolving molecular iodine in a solvent comprising water and a water-miscible polar organic solvent in a water:polar organic solvent ratio of between 5:95 and 50:50 vol.:vol. to provide a solution, wherein the polar organic solvent comprises a water miscible polar protic organic solvent that is independently chosen from: a carboxylic acid, a phenol, an alcohol, and combinations thereof;
(c) Treating the wound dressing substrate with the solution to provide the antimicrobial wound dressing.

In some preferred embodiments, the method of preparing an antimicrobial wound dressing comprises the steps of:
(a) Providing a wound dressing substrate;
(b) Dissolving molecular iodine in a solvent comprising water and a water-miscible polar organic solvent in a water:polar organic solvent ratio of between 5:95 and 50:50 vol.:vol. to provide a solution, wherein the polar organic solvent comprises a water miscible polar protic organic solvent that is independently selected from the group consisting of: a carboxylic acid, a phenol, an alcohol, and combinations thereof;
(c) Treating the wound dressing substrate with the solution to provide the antimicrobial wound dressing.

In preferred embodiments, at least one polar protic organic solvent comprises an alcohol. At least one alcohol may be independently chosen from: a primary alcohol, a secondary alcohol, a tertiary alcohol, and combinations thereof. At least one alcohol may be independently selected from the group consisting of: a primary alcohol, a secondary alcohol, a tertiary alcohol, and combinations thereof. At least one alcohol may preferably comprise a primary alcohol.

At least one alcohol may be independently chosen from: an aliphatic alcohol, an alicyclic alcohol, an aromatic alcohol, and combinations thereof. At least one alcohol may be independently selected from the group consisting of: an aliphatic alcohol, an alicyclic alcohol, an aromatic alcohol, and combinations thereof. At least one alcohol may preferably comprise an aliphatic alcohol. At least one alcohol may be independently selected from: a linear alcohol, a branched alcohol, and combinations thereof. At least one alcohol may preferably comprise a linear alcohol. At least one alcohol may preferably be independently chosen from: a primary alcohol, an aliphatic alcohol, a linear alcohol, and any combinations thereof. At least one alcohol may preferably be one or more of the group consisting of: a primary alcohol, an aliphatic alcohol, a linear alcohol, and any combinations thereof.

The polar organic solvent in step (b) may comprise at least one C1-C15 alcohol, preferably C1-C10, preferably C1-C5 alcohol, or at least one C1-C4 alcohol; preferably wherein at least one said alcohol is independently chosen from: a primary alcohol, an aliphatic alcohol, a linear alcohol, and any combinations thereof.

The polar organic solvent in step (b) may comprise at least one C1-C15 alcohol, preferably C1-C10, preferably C1-C5 alcohol, or at least one C1-C4 alcohol; preferably wherein at least one said alcohol is one or more of the group consisting of: a primary alcohol, an aliphatic alcohol, a linear alcohol, and any combinations thereof.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solvent comprises water and a water miscible polar protic organic solvent comprising at least one C1-C10, preferably C1-C5 alcohol; and
(c) Treating the wound dressing substrate with the solution to provide the antimicrobial wound dressing.

In some preferred embodiments, the method of preparing an antimicrobial wound dressing comprises the steps of:
(a) Providing a wound dressing substrate;
(b) Dissolving molecular iodine in a solvent comprising water and a water-miscible polar organic solvent in a water:polar organic solvent ratio of between 5:95 and 50:50 vol.:vol. to provide a solution, wherein the polar organic solvent comprises a water miscible polar protic organic solvent comprising at least one C1-C10, preferably C1-C5 alcohol;
(c) Treating the wound dressing substrate with the solution to provide the antimicrobial wound dressing.

In some embodiments, at least one alcohol is a mono-ol (i.e. the alcohol contains a single hydroxyl group). At least one alcohol may be substituted with one or more groups independently chosen from: an amine (which may comprise a primary, secondary and/or tertiary amine), a carbonyl-containing group, a halogen atom (which may comprise iodine, bromine, chlorine, and/or fluorine), and combinations thereof. At least one alcohol may be substituted with one or more groups independently selected from the group consisting of: an amine (which may comprise a primary, secondary and/or tertiary amine), a carbonyl-containing group, a halogen atom (which may comprise iodine, bromine, chlorine, and/or fluorine), and combinations thereof. In some preferred embodiments, at least one alcohol is unsubstituted. In some embodiments, at least one alcohol comprises a C1-C15, C1-C10, C1-C5, or C1-C4 alcohol, preferably a mono-ol. At least one alcohol may comprise an unsubstituted C1-C15, C1-C10, C1-C5, or C1-C4 alcohol, preferably a mono-ol.

At least one alcohol may be independently chosen from: methanol, ethanol, a propanol, a butanol, a pentanol, a hexanol, a heptanol, an octanol, and combinations thereof. At least one alcohol may be independently selected from the group consisting of: methanol, ethanol, a propanol, a butanol, a pentanol, a hexanol, a heptanol, an octanol, and combinations thereof. At least one alcohol may be independently chosen from: methanol, ethanol, propanol, isopropanol, 1-butanol, 2-butanol, t-butanol, and combinations thereof. At least one alcohol may be independently selected from the group consisting of: methanol, ethanol, propanol, isopropanol, 1-butanol, 2-butanol, t-butanol, and combinations thereof. At least one alcohol may be independently chosen from: methanol, ethanol, propanol, isopropanol, and combinations thereof. At least one alcohol may be independently selected from the group consisting of: methanol, ethanol, propanol, isopropanol, and combinations thereof. At least one alcohol may preferably comprise ethanol.

In some embodiments, at least 50 wt % of the polar organic solvent in step (b) is an alcohol, or at least 60, 70, 80, or at least 90, 95, 96, 97, 98, or at least 99 wt % of the polar organic solvent in step (b) is an alcohol. In some embodiments, 100 wt % of the polar organic solvent in step (b) is an alcohol. In such embodiments, the alcohol or combination of alcohols may preferably be as described above. At least one alcohol may be independently chosen from: a mono-ol, a primary alcohol, an aliphatic alcohol, a linear alcohol, and any combinations thereof. At least one alcohol may be one or more of the group consisting of: a mono-ol, a primary alcohol, an aliphatic alcohol, a linear alcohol, and any combinations thereof. The or at least one alcohol may preferably be ethanol.

The polar organic solvent may comprise at least one polar aprotic organic solvent. In some embodiments, at least one polar aprotic organic solvent is water miscible. In some embodiments, the polar organic solvent may solely comprise polar aprotic solvent(s). At least one polar aprotic organic solvent may be independently chosen from: a nitrile, a sulfoxide, a ketone, an amide, a phosphoramide, a sulfone, a nitro compound, a carbonate, an ether, as ester, a lactam, a heteroaromatic, and combinations thereof. At least one polar aprotic organic solvent may be independently selected from the group consisting of: a nitrile, a sulfoxide, a ketone, an amide, a phosphoramide, a sulfone, a nitro compound, a carbonate, an ether, as ester, a lactam, a heteroaromatic, and combinations thereof. At least one polar aprotic organic solvent may be independently chosen from: dimethylsulfoxide (DMSO), dimethylformamide (DMF), acetonitrile, acetone, 2-butanone, glyme, diglyme, diethyl ether, dioxane, ethyl acetate, hexamethylphosphoramide (HMPA), hexamethylphosphorustriamide (HMPT), methyl tert-butyl ether, N-methyl-2-pyrrolidinone (NMP), pyridine, tetrahydrofuran, and combinations thereof. At least one polar aprotic organic solvent may be independently selected from the group consisting of: dimethylsulfoxide (DMSO), dimethylformamide (DMF), acetonitrile, acetone, 2-butanone, glyme, diglyme, diethyl ether, dioxane, ethyl acetate, hexamethylphosphoramide (HMPA), hexamethylphosphorustriamide (HMPT), methyl tert-butyl ether, N-methyl-2-pyrrolidinone (NMP), pyridine, tetrahydrofuran, and combinations thereof.

At least one polar aprotic organic solvent may be substituted or unsubstituted. At least one polar aprotic organic solvent may be substituted with one or more groups independently chosen from: an amine (which may comprise a primary, secondary and/or tertiary amine), a halogen atom (which may comprise iodine, bromine, chlorine, and/or fluorine), and combinations thereof. At least one polar aprotic organic solvent may be substituted with one or more groups independently selected from the group consisting of: an amine (which may comprise a primary, secondary and/or tertiary amine), a halogen atom (which may comprise iodine, bromine, chlorine, and/or fluorine), and combinations thereof.

In some embodiments, at least one polar aprotic organic solvent comprises a dipolar aprotic solvent. At least one dipolar aprotic solvent may be independently chosen from: an amide, a sulfoxide, a phosphoramide, a sulfone, a ketone, a nitrile, a nitro compound, a lactam, and combinations thereof. At least one dipolar aprotic solvent may be independently selected from the group consisting of: an amide, a sulfoxide, a phosphoramide, a sulfone, a ketone, a nitrile, a nitro compound, a lactam, and combinations thereof.

In some embodiments, at least one polar aprotic organic solvent comprises a ketone. At least one ketone may be a mono-ketone. In some embodiments, at least one ketone is unsubstituted. At least one ketone may comprise a C1-C10, or C3-C5 ketone, which may be a mono-ketone. At least one ketone may comprise an unsubstituted C1-C10, or C3-C5 ketone, which may be a mono-ketone. At least one ketone may comprise acetone.

In some embodiments, the polar organic solvent comprises at least one polar protic organic solvent and at least one polar aprotic organic solvent. In some embodiments, the polar organic solvent comprises the polar protic solvent and the polar aprotic solvent in a polar protic to polar aprotic solvent ratio of between 1:50 and 50:1, or between 1:40 and 40:1, or between 1:30 and 30:1, or between 1:20 and 20:1, or between 1:10 and 10:1, or between 1:5 and 5:1. The polar organic solvent may comprise the polar protic solvent and the polar aprotic solvent in a polar protic to polar aprotic solvent ratio of between 1:50 and 1:1, or between 1:40 and 1:1, or between 1:30 and 1:1, or between 1:20 and 1:1, or between 1:10 and 1:1, or between 1:5 and 1:1. The polar organic solvent may comprise the polar protic solvent and the polar aprotic solvent in a polar protic to polar aprotic solvent ratio of between 50:1 and 1:1, or between 40:1 and 1:1, or between 30:1 and 1:1, or between 20:1 and 1:1, or between 10:1 and 1:1, or between 5:1 and 1:1. In such embodiments, the polar aprotic solvent may comprise a dipolar aprotic solvent and the polar protic solvent may comprise an alcohol, preferably as described in statements of invention above.

In some embodiments, the solvent in step (b) comprises the polar organic solvent in a total amount of at least 20 wt % of the solvent, or at least 25, 30, 35, 40, 45, or at least 50, 55, 60, 65, 70, 75, 80, 85, or at least 90 wt %. In some embodiments, the solvent in step (b) comprises the polar organic solvent in a total amount of no greater than 95 wt % of the solvent, or no greater than 90, 85, 80, 75, 70, 65, 60, 55, or no greater than 50 wt % of the solvent. The solvent in step (b) may comprise the polar organic solvent in a total amount of between 50-95 wt % of the solvent, or between 60-95, 70-95, or between 75-95, or between 75-85 wt % of the solvent.

In embodiments wherein the step (b) solvent comprises water and a water-miscible polar organic solvent, the water-miscible polar organic solvent may preferably be as described in statements of invention above.

In some embodiments, the solvent in step (b) comprises water in a total amount of at least 1 wt % of the solvent, or at least 2, 3, 4, 5, 6, 7, 8, 9, 10, or at least 15 wt % of the solvent. In some embodiments, the solvent in step (b) comprises water in a total amount of no greater than 50 wt % of the solvent, or no greater than 45, 40, 35, 30, 25, 20, 15, or in a total amount of no greater than 10 wt % of the solvent. The solvent in step (b) may comprise water in a total amount of between 5-50 wt % of the solvent, or between 5-40, 5-30, or between 5-25 wt %, or between 10-25, or between 15-25 wt % of the solvent.

In some embodiments, the solvent in step (b) comprises the polar organic solvent in a total amount of between 50-95 wt % of the solvent, or between 60-95, 70-95, or between 75-95, or between 75-85 wt % of the solvent; and the solvent in step (b) comprises water in a total amount of between 5-50 wt % of the solvent, or between 5-40, 5-30, or between 5-25 wt %, or between 10-25, or between 15-25 wt % of the solvent.

In some embodiments, the solvent in step (b) comprises water and a water-miscible polar organic solvent in a water: polar organic solvent ratio of between 5:95 and 45:55, or between 5:95 and 40:60, or between 5:95 and 35:65, or between 5:95 and 30:70, or between 5:95 and 25:75, or between 5:95 and 20:80, or between 5:95 and 15:85, or between 5:95 and 10:90. The solvent in step (b) may have a water: polar organic solvent ratio of between 10:90 and 50:50, or between 15:85 and 50:50, or between 20:80 and 50:50, or between 25:75 and 50:50, or between 30:70 and 50:50, or between 35:65 and 50:50, or between 40:60 and 50:50, or between 45:55 and 50:50.

In some embodiments, the solvent in step (b) has a water: polar organic solvent ratio of between 5:95 and 35:65, preferably between 10:90 and 30:70, or between 15:85 and 25:75, or preferably of around 20:80.

In some preferred embodiments, the method of preparing an antimicrobial wound dressing comprises the steps of:
(a) Providing a wound dressing substrate;
(b) Dissolving molecular iodine in a solvent comprising water and a water-miscible polar organic solvent in a water:polar organic solvent ratio of between 10:90 and 30:70 vol.:vol. preferably of between 15:85 and 25:75 vol.:vol.;
(c) Treating the wound dressing substrate with the solution to provide the antimicrobial wound dressing.

Such ratios of polar organic solvent to water allow for an even distribution of active iodine to be achieved throughout the wound dressing.

In some embodiments, the solvent in step (b) comprises the polar organic solvent in a total amount of between 50-95 wt % of the solvent, or between 60-95, 70-95, or between 75-95, or between 75-85 wt % of the solvent; and the solvent further comprises water in a water: polar organic solvent ratio of between 5:95 and 35:65, or between 10:90 and 30:70, or between 15:85 and 25:75.

In some embodiments, the solvent in step (b) comprises water in a total amount of between 5-50 wt % of the solvent, or between 5-40, 5-30, or between 5-25 wt %, or between 10-25, or between 15-25 wt % of the solvent; and the solvent further comprises a polar organic solvent in a water: polar organic solvent ratio of between 5:95 and 35:65, or between 10:90 and 30:70, or between 15:85 and 25:75.

In embodiments wherein step (b) comprises dissolving molecular iodine and at least one halide-containing species in the solvent, at least one halide-containing species may be independently chosen from: a fluoride-containing species, a chloride-containing species, a bromide containing species, an iodide-containing species, and combinations thereof. In embodiments wherein step (b) comprises dissolving molecular iodine and at least one halide-containing species in the solvent, at least one halide-containing species may be independently selected from the group consisting of: a fluoride-containing species, a chloride-containing species, a bromide containing species, an iodide-containing species, and combinations thereof. In some preferred embodiments, at least one halide-containing species may be independently chosen from: a chloride-containing species, an iodide containing species, and combinations thereof. In some preferred embodiments, at least one halide-containing species may be independently selected from the group consisting of: a chloride-containing species, an iodide containing species, and combinations thereof. In preferred embodiments, at least one halide-containing species comprises at least one iodide-containing species. In some embodiments, at least one halide-containing species comprises a halide of formula X⁻, wherein X is as described for the first aspect of the invention above. Preferably, the halide from the halide-containing species binds with molecular iodine to provide a species of Formula (I), as described for the first aspect of the invention above.

In some embodiments, step (b) comprises dissolving more than one different halide-containing species in the solvent. Step (b) may comprise dissolving 2 or at least 2 different halide-containing species in the solvent, or 3 or at least 3, or 4 different halide-containing species in the solvent. Step (b) may comprise dissolving no greater than 3 different halide-containing species in the solvent, or no greater than 2 different halide-containing species in the solvent. In some embodiments, step (b) comprises dissolving a single halide-containing species in the solvent.

In some embodiments, at least one halide-containing species comprises at least one halide-containing salt. At least one halide-containing salt may comprise a counter-cation that is independently chosen from: an alkali metal, an alkaline earth metal, a group III metal, and a transition metal. At least one halide-containing salt may comprise a counter-cation that is independently selected from the group consisting of: an alkali metal, an alkaline earth metal, a group III metal, and a transition metal. At least one halide-containing salt may comprise a counter-cation that is independently chosen from: an alkali metal, and an alkaline earth metal. At least one halide-containing salt may comprise a counter-cation that is independently selected from the group consisting of: an alkali metal, and an alkaline earth metal. At least one halide-containing species may preferably comprise at least one alkali metal halide salt. At least one alkali metal halide salt may be independently chosen from: a lithium halide, a potassium halide, a sodium halide, and combinations thereof. At least one alkali metal halide salt may be independently selected from the group consisting of: a lithium halide, a potassium halide, a sodium halide, and combinations thereof. At least one alkali metal halide salt may preferably comprise at least one alkali metal iodide salt, which may comprise sodium iodide.

In some embodiments, step (b) comprises dissolving at least a molar equivalent of the halide-containing species relative to molecular iodine in the solvent. In some embodiments, step (b) comprises dissolving the halide-containing species in the solvent in amount of at least 1.5 times the amount of molecular iodine, or at least 2, 2.5, 3, 3.5, 4, 4.5, 5, 5.5, 6, 6.5, 7, 7.5, 8, 8.5, 9, 9.5, or at least 10 times the amount of molecular iodine. In some embodiments, step (b) comprises dissolving the halide-containing species and the molecular iodine in the solvent in a halide-containing species to molecular iodine molar ratio of between 1:1 and 10:1, or between 1:1 and 8:1, 1:1 and 6:1, 1:1 and 4:1, or between 1:1 and 2:1.

In some embodiments, the solution prepared in step (b) has a molecular iodine concentration of at least 1 mM, or at least 2, 3, 4, 5 mM, or at least 10, 15, 20, or at least 25 mM, or at least 30, 35, 40, 45, 50, 55, 60, 65, 70, or at least 75 mM. In some embodiments, the solution prepared in step (b) has a molecular iodine concentration of no greater than 200 mM, or no greater than 195, 190, 185, 180, 175, 170, 165, 160, 155, or no greater than 150 mM, or no greater than 140, 130, 120, 110, 100, 90, or no greater than 80 mM. In some embodiments, the solution prepared in step (b) has a molecular iodine concentration of between 5-155 mM, or between 15-145, 25-135, 35-125, or between 45-115 mM, or between 55-105, 65-95, or between 75-85 mM.

In some embodiments, the solution prepared in step (b) has a molecular iodine concentration of between 5-155 mM, or between 15-145, 25-135, 35-125, or between 45-115 mM, or between 55-105, 65-95, or between 75-85 mM; and the molecular iodine is dissolved in a solvent comprising water and a polar organic solvent in a water: polar organic solvent ratio of between 5:95 and 35:65, or between 10:90 and 30:70, or between 15:85 and 25:75.

In some embodiments, the solution prepared in step (b) has a molecular iodine concentration of between 5-155 mM, or between 15-145, 25-135, 35-125, or between 45-115 mM, or between 55-105, 65-95, or between 75-85 mM; and the molecular iodine is dissolved in a solvent comprising water and a polar organic solvent, wherein the polar organic solvent is present in a total amount of between 50-95 wt % of the solvent, or between 60-95, 70-95, or between 75-95, or between 75-85 wt % of the solvent, and the water is present in a total amount of between 5-50 wt % of the solvent, or between 5-40, 5-30, or between 5-25 wt %, or between 10-25, or between 15-25 wt % of the solvent.

In some embodiments, the solution prepared in step (b) has a total halide-containing species concentration of at least 1 mM, or at least 2, 3, 4, 5, 10, 20, 30, 40, 50, 60, 70, 80, 90, 100, or at least 105 mM. In some embodiments, the solution prepared in step (b) has a total halide-containing species concentration of no greater than 500 mM, or no greater than 475, 450, 425, 400, 375, 350, 325, 300, 275, 250, 225, 200, 175, 150, 125, 100, 90, or no greater than 80 mM. In some embodiments, the solution prepared in step (b) has a total halide-containing species concentration of between 5-205 mM, or between 15-195, 25-185, 35-175, 45-165, 55-155, 65- 145, 75-135, 85-125, 95-115, or between 100-110 mM.

In some embodiments, the solution prepared in step (b) has a total halide-containing species concentration of between 5-205 mM, or between 15-195, 25-185, 35-175, 45-165, 55-155, 65- 145, 75-135, 85-125, 95-115, or between 100-110 mM; and the halide-containing species is dissolved in a solvent comprising water and a polar organic solvent in a water:polar organic solvent ratio of between 5:95 and 35:65, or between 10:90 and 30:70, or between 15:85 and 25:75.

In some embodiments, the solution prepared in step (b) has a molecular iodine concentration of between 5-155 mM, or between 15-145, 25-135, 35-125, or between 45-115 mM, or between 55-105, 65-95, or between 75-85 mM; and the solution has a total halide-containing species concentration of between 5-205 mM, or between 15-195, 25-185, 35-175, 45-165, 55-155, 65- 145, 75-135, 85-125, 95-115, or between 100-110 mM.

In some embodiments, the solution prepared in step (b) has a total halide-containing species concentration of between 5-205 mM, or between 15-195, 25-185, 35-175, 45-165, 55-155, 65- 145, 75-135, 85-125, 95-115, or between 100-110 mM; and the halide-containing species is dissolved in a solvent comprising water and a polar organic solvent, wherein the polar organic solvent is present in a total amount of between 50-95 wt % of the solvent, or between 60-95, 70-95, or between 75-95, or between 75-85 wt % of the solvent, and the water is present in a total amount of between 5-50 wt % of the solvent, or between 5-40, 5-30, or between 5-25 wt %, or between 10-25, or between 15-25 wt % of the solvent.

In some embodiments, the solution prepared in step (b) has a molecular iodine concentration of between 5-155 mM, or between 15-145, 25-135, 35-125, or between 45-115 mM, or between 55-105, 65-95, or between 75-85 mM; and the solution has a total halide-containing species concentration of between 5-205 mM, or between 15-195, 25-185, 35-175, 45-165, 55-155, 65- 145, 75-135, 85-125, 95-115, or between 100-110 mM; and the molecular iodine and halide-containing species are dissolved in a solvent comprising water and a polar organic solvent, wherein the polar organic solvent is present in a total amount of between 50-95 wt % of the solvent, or between 60-95, 70-95, or between 75-95, or between 75-85 wt % of the solvent, and the water is present in a total amount of between 5-50 wt % of the solvent, or between 5-40, 5-30, or between 5-25 wt %, or between 10-25, or between 15-25 wt % of the solvent.

In some embodiments, the solution prepared in step (b) has a total halide-containing species concentration of between 1 and 10 times the molecular iodine concentration, or between 1 and 8 times, between 1 and 6 times, between 1 and 4 times, or between 1 and 2 times the molecular iodine concentration.

In some embodiments, the solution prepared in step (b) comprises at least one halide-containing species and molecular iodine in a total halide-containing species concentration of between 1 and 10 times the molecular iodine concentration, or between 1 and 8 times, between 1 and 6 times, between 1 and 4 times, or between 1 and 2 times the molecular iodine concentration; and the molecular iodine and halide-containing species are dissolved in a solvent comprising water and a polar organic solvent in a water: polar organic solvent ratio of between 5:95 and 35:65, or between 10:90 and 30:70, or between 15:85 and 25:75.

In some embodiments, the solution prepared in step (b) has a pH of at least 2, or at least 3, 4, 5, 6, or at least 7. In some embodiments, the solution prepared in step (b) has a pH of no greater than 12, or no greater than 11, 10, or no greater than 9. In some embodiments, the solution prepared in step (b) has a pH of between 3-10, or 4-10, or 4-9, or preferably between 5-9, 6-9, or between 7-9.

In some preferred embodiments, the method of preparing an antimicrobial wound dressing comprises the steps of:
(a) Providing a wound dressing substrate;
(b) Dissolving molecular iodine and at least one halide-containing species in a solvent to provide a solution, wherein the solution has a pH of between 4-10; and
(c) Treating the wound dressing substrate with the solution to provide the antimicrobial wound dressing.

In some preferred embodiments, the method of preparing an antimicrobial wound dressing comprises the steps of:
(a) Providing a wound dressing substrate;
(b) Dissolving molecular iodine in a solvent comprising water and a water-miscible polar organic solvent in a water:polar organic solvent ratio of between 5:95 and 50:50 vol.:vol. to provide a solution, wherein the solution has a pH of between 4-10;
(c) Treating the wound dressing substrate with the solution to provide the antimicrobial wound dressing.

In some embodiments, step (b) may comprise adding a pH adjustment agent to the solution. The pH adjustment agent may comprise at least one acid, which may comprise a strong acid. The acid may comprise a mineral acid. In some embodiments, the acid may be independently chosen from: hydrochloric acid, hydriodic acid, perchloric acid, nitric acid, sulfuric acid, phosphoric acid, and combinations thereof. In some embodiments, the acid may be independently selected from the group consisting of: hydrochloric acid, hydriodic acid, perchloric acid, nitric acid, sulfuric acid, phosphoric acid, and combinations thereof. Optionally the acid may be or comprise hydrochloric acid. In some embodiments, the pH adjustment agent may comprise at least one base, which may comprise a strong base. At least one base may comprise an inorganic base. At least one inorganic base may be independently chosen from: a hydroxide base, a carbonate base, a bicarbonate base, and combinations thereof. At least one inorganic base may be independently selected from the group consisting of: a hydroxide base, a carbonate base, a bicarbonate base, and combinations thereof. In some preferred embodiments, at least one base is a hydroxide base. At least one hydroxide base may be independently chosen from: an alkali metal hydroxide, an alkaline earth metal hydroxide, a group III metal hydroxide, a transition metal hydroxide, and combinations thereof. At least one hydroxide base may be independently selected from the group consisting of: an alkali metal hydroxide, an alkaline earth metal hydroxide, a group III metal hydroxide, a transition metal hydroxide, and combinations thereof. At least one hydroxide base may preferably comprise an alkali metal hydroxide and/or an alkaline earth metal hydroxide. In some embodiments, at least one base comprises an alkali metal hydroxide that is independently chosen from: lithium hydroxide, sodium hydroxide, potassium hydroxide, and combinations thereof. In some embodiments, at least one base comprises an alkali metal hydroxide that is independently selected from the group consisting of: lithium hydroxide, sodium hydroxide, potassium hydroxide, and combinations thereof.

In some embodiments, step (b) further comprises the step of adding a pH buffer to the solution. The pH buffer may be added after dissolving the molecular iodine in the solvent. The pH buffer may comprise a mixture of a weak acid and its conjugate base. The pH buffer may be independently chosen from: a citric acid buffer, an acetic acid buffer, a phosphate buffer, a carbonate-bicarbonate buffer, a succinate buffer, a CHES buffer, and combinations thereof. The pH buffer may be independently selected from the group consisting of: a citric acid buffer, an acetic acid buffer, a phosphate buffer, a carbonate-bicarbonate buffer, a succinate buffer, a CHES buffer, and combinations thereof.

In some embodiments, step (b) further comprises the step of adding at least one chelant to the solution. At least one chelant may be or comprise a metal ion complexing agent. At least one chelant may comprise a multidentate ligand. At least one chelant may comprise at least one nitrogen donor and/or at least one oxygen donor. At least one chelant may comprise at least one oxygen donor and may be independently chosen from: citrate, oxalate, phosphate, phosphonate, and combinations thereof. At least one chelant may comprise at least one oxygen donor and may be independently selected from the group consisting of: citrate, oxalate, phosphate, phosphonate, and combinations thereof. In some embodiments, at least one chelant comprises both nitrogen and oxygen donors. At least one chelant may be independently chosen from: ethylenediaminetetraacetic acid (EDTA), tetraxetan (DOTA), diethylenetriaminepentaacetic acid (DTPA), ethylene glycol-bis(β-aminoethyl ether)-N,N,N′,N′-tetraacetic acid (EGTA), and combinations thereof. At least one chelant may be independently selected from the group consisting of: ethylenediaminetetraacetic acid (EDTA), tetraxetan (DOTA), diethylenetriaminepentaacetic acid (DTPA), ethylene glycol-bis(β-aminoethyl ether)-N,N,N′, N′-tetraacetic acid (EGTA), and combinations thereof. In some embodiments, step (b) comprises adding the chelant to the solution in a total amount of between 0.01-5 wt % of the solution, or between 0.05-2.5 wt %, or between 0.1-0.8 wt % of the solution, or between 0.2-0.7, 0.3-0.6, or between 0.4-0.5 wt % of the solution.

In some embodiments, the solution prepared in step (b) has an ionic strength of at least 0.25 mM, or at least 0.5, 0.75, 1, 2, 3, 4, 5, 6, or at least 7 mM. The solution prepared in step (b) may have an ionic strength of no greater than 1000 mM, or no greater than 900, 800, or no greater than 700, 600, 500, 400, 300, 200, 100, or no greater than 90, 80, 70, 60, 50, 40, 30, 20, or no greater than 10 mM. In some embodiments, the solution prepared in step (b) has an ionic strength of between 1 and 1000 mM.

Treating the wound dressing substrate, preferably which contains at least one water-swellable polymer, with solutions having such ionic strengths prevents premature and irreversible swelling of the substrate polymer. However, such a solution allows for reversible swelling to occur, which assists the incorporation of active iodine into the wound dressing whilst maintaining the substrate structure.

In some embodiments, the solution prepared in step (b) has a pH of between 3-10, or 4-10, or 4-9, or preferably between 4.5-8.5, or more preferably between 5-8; and the solution has an ionic strength of between 1 and 1000 mM.

In some embodiments, step (b) further comprises the step of adding at least one salt to the solution, preferably an inert salt. At least one salt may be added to increase the ionic strength of the solution to values as stated above. At least one salt may comprise a monocharged cation and/or anion. At least one salt may comprise a di- or multicharged cation or anion. At least one salt may comprise a cation independently chosen from: an alkali metal cation, an alkaline earth metal cation, a group III metal cation, a transition metal cation, an ammonium cation, an aromatic nitrogen-based cation, and combinations thereof. At least one salt may comprise a cation independently selected from the group consisting of: an alkali metal cation, an alkaline earth metal cation, a group III metal cation, a transition metal cation, an ammonium cation, an aromatic nitrogen-based cation, and combinations thereof. At least one salt may comprise a cation that is independently chosen from: ammonium, calcium, iron, magnesium, potassium, pyridinium, quaternary ammonium, sodium, copper, silver, zinc, and combinations thereof. At least one salt may comprise a cation that is independently selected from the group consisting of: ammonium, calcium, iron, magnesium, potassium, pyridinium, quaternary ammonium, sodium, copper, silver, zinc, and combinations thereof. At least one salt may comprise an inorganic and/or organic anion. At least one salt may comprise an anion that is independently chosen from: acetate, carbonate, chloride, bromide, iodide, citrate, cyanide, fluoride, nitrate, nitrite, oxide, phosphate, sulfate, allantoinate, borate, glycerolate, lactate, laurate, deoxycholate, salicylate, aminobenzoate, aminosalicylate, and combinations thereof. At least one salt may comprise an anion that is independently selected from the group consisting of: acetate, carbonate, chloride, bromide, iodide, citrate, cyanide, fluoride, nitrate, nitrite, oxide, phosphate, sulfate, allantoinate, borate, glycerolate, lactate, laurate, deoxycholate, salicylate, aminobenzoate, aminosalicylate, and combinations thereof. In some embodiments, the sum of the moduli of the charges of the constituent ions of at least one salt added in step (b) is 2 or at least 2, 3 or at least 3, or 4 or at least 4. The sum of the moduli of the charges of the constituent ions is calculated for potassium nitrate, by way of example, as follows: KNO₃→K⁺ (modulus of charge=1) and NO₃⁻ (modulus of charge=1)→sum of the moduli=1+1=2.

In some embodiments, at least one salt is a silver salt. In some embodiments, at least one salt is independently chosen from: silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver aminobenzoate, silver aminosalicylate, nanocrystalline silver, a silver oxysalt, and combinations thereof. In some embodiments, at least one salt is independently selected from the group consisting of: silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver aminobenzoate, silver aminosalicylate, nanocrystalline silver, a silver oxysalt, and combinations thereof.

In some embodiments, steps (a) and (b) and (c) may be performed simultaneously. The method may comprise treating the wound dressing substrate with the solution of step (b) during formation of the wound dressing substrate. The method may comprise treating the wound dressing substrate with the solution of step (b) during a fibre-forming stage of the wound dressing formation. In such embodiments, the iodine from the solution may be incorporated within the wound dressing substrate, preferably as described for the first aspect of the invention above.

In other embodiments, step (c) is performed after step (a). The method may comprise treating the wound dressing substrate with the solution of step (b) after formation of the wound dressing substrate. In such embodiments, the iodine from the solution may be incorporated within the wound dressing substrate and/or adsorbed to a surface of the wound dressing substrate, preferably as described for the first aspect of the invention above.

In some embodiments, step (c) comprises the step of moistening the wound dressing substrate with a solvent before, during, and/or after treatment of the wound dressing substrate with the solution prepared in step (b). The moistening solvent may be the same as the solvent used in step (b). Statements of invention relating to the solvent of step (b) may also be applied to the moistening solvent. Alternatively, the moistening solvent may be different to the solvent used in step (b).

The step of treating the wound dressing substrate with the solution in step (c) may involve any suitable treatment method known in the art. In some embodiments, the step of treating the wound dressing substrate with the solution involves one or more treatment methods independently chosen from: submersion, spray coating, soaking, dipping, wetting, and combinations thereof. In some embodiments, the step of treating the wound dressing substrate with the solution involves one or more treatment methods independently selected from the group consisting of: submersion, spray coating, soaking, dipping, wetting, and combinations thereof.

In some embodiments, step (c) comprises treating the wound dressing substrate with the solution for a total time of at least 5 seconds, or at least 10, 20, 30, 40, or at least 50 seconds, or at least 1 minute, or at least 2, 3, 4, or at least 5 minutes, or at least 10, 20, 30, 40, or at least 50 minutes, or at least 1 hour, or at least 1.5, 2, 2.5, 3, 3.5, or at least 4 hours. Step (c) may comprise treating the wound dressing substrate with the solution for a total time of no greater than 1 week, or no greater than 6 days, or no greater than 5, 4, 3, 2, or no greater than 1 day, or no greater than 20 hours, 19, 18, 17, 16, 15, 14, 13, 12, 11, 10, 9, 8, 7, 6, or no greater than 5 hours.

In some embodiments, step (c) comprises treating the wound dressing substrate with the solution at a temperature of at least 5° C., or at least 10, 15, 20, or at least 25° C. Step (c) may comprise treating the wound dressing substrate with the solution at a temperature of no greater than 100° C., or no greater than 90, 80, 70, 60, 50, or no greater than 40° C.

In some embodiments, step (c) comprises the step of sonically agitating the wound dressing substrate. Said step may be performed before, during and or after treatment of the wound dressing substrate with the solution. The sonic agitation step may preferably be performed before and/or during treatment of the substrate with the solution. Sonic agitation can help facilitate easier incorporation of the solution/active iodine into the internal structure of the wound dressing substrate.

In some embodiments, step (c) further comprises the step of treating the wound dressing substrate with a post-treatment solution after treatment with the solution of step (b). The post-treatment wash solution may comprise a polar organic solvent, preferably as described in statements relating to the solution prepared in step (b) above, which may be present in an amount of at least 95 wt. %, or at least 96, 97, 98, or at least 99 wt. %. In some embodiments, the post-treatment solution may comprise water, which may be present in an amount of from 0.01-1.9 wt. %, or from 0.1-1.5 wt. %, or from 0.5-1 wt. %. In some embodiments, the post-treatment solution may comprise at least one surfactant, which may be present in a total amount from 0.1-2 wt. %, or from 1-1.5 wt. %. At least one surfactant may be independently chosen from: a nonionic surfactant, an anionic surfactant, a cationic surfactant, a zwitterionic surfactant, and combinations thereof. At least one surfactant may be independently selected from the group consisting of: a nonionic surfactant, an anionic surfactant, a cationic surfactant, a zwitterionic surfactant, and combinations thereof. At least one surfactant may preferably comprise a nonionic surfactant, which may be independently chosen from: a polysorbate, a Triton-based surfactant, a poloxamer, a sorbitan alkyl ester, and combination thereof. At least one surfactant may preferably comprise a nonionic surfactant, which may be independently selected from the group consisting of: a polysorbate, a Triton-based surfactant, a poloxamer, a sorbitan alkyl ester, and combination thereof. At least one surfactant may comprise a polysorbate. At least one surfactant may comprise a nitrogen-containing surfactant, which may be a cationic surfactant. At least one nitrogen-containing surfactant may comprise a quaternary ammonium surfactant. At least one nitrogen-containing surfactant may be independently chosen from: a benzethonium surfactant, a benzalkonium surfactant, a monoalkyl trimethylammonium surfactant, a dimethyl dialkylammonium surfactant, a trialkyl methylammonium surfactant, an alkylpyridinium surfactant, and combinations thereof. At least one nitrogen-containing surfactant may be independently selected from the group consisting of: a benzethonium surfactant, a benzalkonium surfactant, a monoalkyl trimethylammonium surfactant, a dimethyl dialkylammonium surfactant, a trialkyl methylammonium surfactant, an alkylpyridinium surfactant, and combinations thereof. The nitrogen-containing surfactant may be present in a total amount of between 0.01-1 wt % of the post-treatment solution, or between 0.02-0.75 wt %, 0.03-0.5 wt %, or between 0.05-0.2 wt % of the post-treatment solution. In some embodiments, the nitrogen-containing surfactant may be present in a total amount of no greater than 1 wt % of the post-treatment solution, or no greater than 0.75 wt %, or no greater than 0.5 wt % of the post-treatment solution.

In some embodiments, the method further comprises the step of drying the wound dressing substrate after treatment with the solution of step (b) or with a post-treatment solution. The step may comprise partially or fully drying the substrate. The method may comprise the step of compressing, squeezing or pressing the substrate to expel moisture. Compression, squeezing, or pressing can assist drying and drive impregnation of the substrate with an active iodine species. In some embodiments, the drying step comprises one or more chosen from: air drying, oven drying, mechanical pressing, centrifugation, vacuum extraction, and combinations thereof. In some embodiments, the drying step comprises one or more of the group consisting of: air drying, oven drying, mechanical pressing, centrifugation, vacuum extraction, and combinations thereof. The drying step may be performed at a temperature of between 20-140° C., or between 30-130, 40-120, or between 50-110° C. The drying step may be performed for a total duration of between 0.5-30 hours, or between 5-25, 10-20, or between 14-18 hours. In some embodiments, the drying step is performed for a total duration of no greater than 10 hours, or preferably no greater than 8 hours, or no greater than 6, 4, or no greater than 2 hours. In some embodiments, the drying step may comprise oven drying the wound dressing substrate at a temperature of between 15-50° C., or between 20-45, or between 25-40° C., for a duration of between 10-20 hours, or between 14-18 hours.

According to fourth aspect of the invention, there is provided an antimicrobial wound dressing obtainable by a method comprising the steps of:
(a) Providing a wound dressing substrate;
(b) Dissolving molecular iodine and at least one halide-containing species in a solvent to provide a solution; and
(c) Treating the wound dressing substrate with the solution to provide the antimicrobial wound dressing.

The method of obtaining the antimicrobial wound dressing of the fourth aspect of the invention is preferably the method of the second aspect of the invention. Statements of invention relating to the method of the second aspect of the invention above may also be applied to the fourth aspect of the invention.

According to a fifth aspect of the invention, there is provided an antimicrobial wound dressing obtainable by a method comprising the steps of:
(a) Providing a wound dressing substrate;
(b) Dissolving molecular iodine in a solvent comprising water and a water-miscible polar organic solvent in a water:polar organic solvent ratio of between 5:95 and 50:50 vol.:vol. to provide a solution; and
(c) Treating the wound dressing substrate with the solution to provide the antimicrobial wound dressing.

The method of obtaining the antimicrobial wound dressing of the fifth aspect of the invention is preferably the method of the third aspect of the invention. Statements of invention relating to the method of the third aspect of the invention above may also be applied to the fifth aspect of the invention.

The following statements apply to the fourth and fifth aspects of the invention.

The antimicrobial wound dressings of the fourth and fifth aspects of the invention are preferably the antimicrobial wound dressing of the first aspect of the invention. Statements of invention above relating to the antimicrobial wound dressing of the first aspect of the invention or to any components thereof may also be applied to the fourth and fifth aspects of the invention. Other statements of invention relating to the first aspect of the invention may also be applied mutatis mutandis to the fourth and fifth aspects of the invention. Other statements of invention above relating to any aspect of the invention may also be applied mutatis mutandis to the fourth and fifth aspects of the invention.

According to a sixth aspect of the invention, there is provided a method of treating a wound comprising the step of applying to the wound an antimicrobial wound dressing of the first, fourth, or fifth aspects of the inventions.

Statements of invention above relating to the wound dressings of the first, fourth, and fifth aspects of the invention or to any components thereof may also be applied to the sixth aspect of the invention. Statements of invention above relating to the methods of the second and third aspects of the invention may also be applied to the sixth aspect of the invention.

In some embodiments, the wound is an open wound and may be independently chosen from: an incision, a laceration, a graze, an avulsion, a puncture wound, a penetration wound, a gunshot wound, a critical wound, and combinations thereof. In some embodiments, the wound is an open wound and may be independently selected from the group consisting of: an incision, a laceration, a graze, an avulsion, a puncture wound, a penetration wound, a gunshot wound, a critical wound, and combinations thereof. In some embodiments, the wound is independently chosen from: a cut, a graze, a burn, and an ulcer. In some embodiments, the wound is independently selected from the group consisting of: a cut, a graze, a burn, and an ulcer.

### DETAILED DESCRIPTION OF THE INVENTION

In order that the invention may be more clearly understood embodiments thereof will now be described, by way of example only.

### Example 1 (Direct addition of Molecular Iodine)

A first embodiment of a wound dressing of the invention was prepared as follows:
(a) A fibrous sodium carboxymethylcellulose stitched AQUACEL® Extra wound dressing substrate sample was provided having dimensions of 5×5 cm. The dressing substrate sample was formed from two layers: (i) a nonwoven layer; and (ii) a needle-punched fabric layer. Each layer had a nominal basis weight of 70 g/m² and the layers were stitch-bonded together with a cellulose thread. The fibres forming the fabric were a low degree of substitution (nominally 0.3) sodium carboxymethylcellulose fibre of approximately 1.2 dTex.
(b) Molecular iodine was dissolved in an 80:20 ethanol:water solvent mixture to provide a solution having a molecular iodine concentration of 79 mM.
(c) A total of 5 mL of the solution prepared in step (b) was directly added to the wound dressing substrate by pipetting five 1 mL aliquots onto the substrate. The substrate was thereafter left to air dry to provide an antimicrobial wound dressing of the invention. Use of such a step (b) solvent mixture allowed for an equal distribution of iodine throughout the wound dressing substrate. This was made evident by the fact that the wound dressing substrate had an equal, uniform black colouration throughout the fibrous substrate. However, non-CMC stitched cellulose of the substrate remained uncoloured.

Iodine extraction from the prepared wound dressing was thereafter assessed.

15 mL of saline were added to the prepared antimicrobial wound dressing. The wound dressing was thereafter covered and placed in an oven for approximately 30 minutes at 37° C. (to mimic the internal temperature of the human body). The saline solution discoloured due to a release of iodine into solution. The wound dressing sample was removed from the oven and the saline/iodine solution was transferred to a container for analysis. Another 15 mL batch of saline was then added to the wound dressing and the above steps were repeated until there was no more iodine visually detectable from an extraction. The amount of iodine release by the dressing was then calculated via titration using sodium thiosulfate and starch indicator.

The results found that 4.7% of the iodine added to the wound dressing substrate was released upon extraction. The wound dressing substrate mostly decoloured upon iodine extraction and appeared largely identical to the dressing substrate prior to treatment with iodine—this suggested that iodine formed a largely physical interaction with the wound dressing substrate in the dressing, as opposed to being chemically bound.

### Example 2 (Addition of Iodine in an Excess of Iodide at an Acidic pH)

A second embodiment of a wound dressing of the invention was prepared as follows:
(a) Step (a) was performed as for Example 1 above.
(b) Molecular iodine and an excess of potassium iodide were dissolved in an 80:20 ethanol:water solvent mixture to provide a solution having a molecular iodine concentration of 79 mM. The solution had a pH of 5.35.
(c) Step (c) was performed as for Example 1 above. In this case, however, both the fibrous substrate and non-CMC stitched sections of the substrate retained a uniform black colouration post-drying.

Iodine extraction from the prepared wound dressing was thereafter assessed as for Example 1 above.

The results found that 40.4% of the iodine added to the wound dressing substrate was released upon extraction, which gave an average iodine release per unit area of the wound dressing of around 1.6 mg/cm². The fibrous wound dressing substrate remained black even upon iodine extraction, which suggested that iodine had chemically bound or formed a stronger physical interaction with the wound dressing substrate compared to the dressing of Example 1.

It is believed that the releasable iodine is present in the wound dressing as a triiodide complex ion ([I—I—I]⁻), which forms through binding of molecular iodine with iodide ions from the added potassium iodide. The triiodide is believed to be complexed with a non-polymeric counterion (such as a potassium cation) and/or complexed with the wound dressing substrate. The presence of triiodide in such states allows for good retainment of molecular iodine in the dressing in dry conditions, whilst also providing excellent molecular iodine release properties in moist, wound-like conditions.

### Example 3 (Effect of pH in Excess Iodide)

Third, fourth and fifth embodiments of wound dressings of the invention were prepared as for Example 2 above. However, the pH of the solution in step (b) of the method was raised by the addition of 2 M sodium hydroxide to obtain final solution pH values of 7 (third embodiment), 8 (fourth embodiment) and 9.5 (fifth embodiment).

The samples prepared at pH 7 and 8 appeared largely similar to the sample prepared in Example 2. Samples produced at pH 9 appeared slightly paler.

Iodine extraction from the wound dressings was assessed as for Example 1 above and results are displayed in Table 1 below.

TABLE 1

| pH (*) | Percentage | Iodine release per |
|---|---|---|
| data from | iodine | unit area of |
| Example 2 | extracted | dressing (mg/cm²) |
| 5.35 (*) | 40.4% | 1.6 |
| 7 | 44.1% | 1.6 |
| 8 | 47.3% | 1.9 |
| 9.5 | 30.8% | 1.2 |

Iodine release was good at all measured pH values. However, whilst increasing the pH from 5.35 to 8 saw an increase in release, further increasing the pH resulted in a quite significant drop in measured release properties.

In all cases, the fibrous wound dressing substrates retained colouration even upon iodine extraction, which again suggested that iodine had chemically bound or formed a strong physical interaction with the wound dressing substrate.

### Example 4

A sixth embodiment of a wound dressing of the invention was prepared as follows:
(a) Step (a) was performed as for Example 1 above.
(b) Equimolar amounts of molecular iodine and potassium iodide were dissolved in an 80:20 ethanol:water solvent mixture to provide a solution having a molecular iodine concentration of 7.9 mM. The solution had a pH of 7, which suggested that an excess of iodide as used in Example 2 results in a more acidic pH.
(c) Step (c) was performed as for Example 1 above. Appearance of the substrate post-drying was as for Example 1 above.

Iodine extraction from the prepared wound dressing was thereafter assessed as for Example 1 above.

The results found that 21.2% of the iodine added to the wound dressing substrate was released upon extraction, which gave an average iodine release per unit area of the wound dressing of around 0.085 mg/cm². Post-extraction, the wound dressing substrate decoloured as for Example 1 above, which again suggested that iodine formed a largely physical interaction with the wound dressing substrate in the dressing, as opposed to being chemically bound. However, even with the reduced amount of iodine added, good iodine release was observed—this is likely due to the co-addition of potassium iodide, which had a significant effect despite only being added in an equimolar amount to the molecular iodine.

### Example 5 Microbiological Testing

### Standard Iodine Loadings

Three different batches of dressing samples were prepared. All the samples had 2'2 cm fibrous sodium carboxymethylcellulose stitched substrates. Measures were taken throughout to minimize the effects of initial or procedure-introduced bioburden.

Batch 1 (control): control sample batch. 1 mL of ethanol was added to each sample to sterilise them. The samples were left to dry overnight.

Batch 2 (control): iodide sample batch. A 20 mL solution was prepared using ethanol and water (80:20), and potassium iodide. 1 mL of solution was added to each sample. The samples were left to dry overnight.

Batch 3 (inventive samples): iodine sample batch. A 20 mL solution was prepared using ethanol and water (80:20), potassium iodide and iodine. 1 mL of sample was added to each dressing. The samples were left to dry overnight.

Masses of excipients used in the Batch 1 and 2 treatment solutions along with their respective pH values are displayed in Table 2 below.

TABLE 2

| | Batch 2 | Batch 3 |
|---|---|---|
| | solution | solution |
| Mass of molecular | 0 | 391 |
| iodine (mg) | | |
| Mass of potassium | 352 | 352 |
| iodide (mg) | | |
| pH | 7.10 | 7.51 |

Masses of molecular iodine and potassium iodide added per sample are displayed in Table 3 below.

TABLE 3

| | Batch 1 | Batch 2 | Batch 3 |
|---|---|---|---|
| | sample | sample | sample |
| Mass of molecular | 0 | 0 | 20 |
| iodine (mg) | | | |
| Mass of potassium | 0 | 18 | 18 |
| iodide (mg) | | | |

Zone on inhibition (ZOI) assays were performed on the following bacteria.

P. aeruginosa

The ZOI assay conducted on the gram-negative bacteria, P. aeruginosa, showed batch 3 samples of the invention to have excellent antimicrobial activity. There was a significant zone of inhibition, as well as a clear circle of iodine release shown around the sample. The control batch 1 samples displayed no antimicrobial activity, with evident bacterial growth on the wound dressing substrate. Batch 2 control iodide samples displayed a slight zone of inhibition, as well as no bacterial growth on the sample—however, zone of inhibition was far lower than for the batch 3 samples of the invention.

ZOI results for P. aeruginosa are displayed in Table 4 below.

TABLE 4

| | Mean zone | |
|---|---|---|
| | of inhibition | Standard |
| Dressing | (mm) | deviation |
| Batch 1 | 0 | 0 |
| Batch 2 | 3.3 | 1.4 |
| Batch 3 | 42 | 0.95 |

S. aureus

The ZOI assay conducted on the gram-positive bacteria, S. aureus, showed a very strong zone of inhibition for the batch 3 samples of the invention. There was a full release of iodine resulting in batch 3 dressings appearing colourless post-treatment. Further, all bacteria on the agar plate were killed. For control batch 1 samples—whilst there was no visible bacterial growth on the sample, there was clear growth around and underneath it. Batch 2 controls also had no bacterial growth on the samples and displayed a small zone of inhibition.

ZOI results for S. aureus are displayed in Table 5 below.

TABLE 5

| | Mean zone | |
|---|---|---|
| | of inhibition | Standard |
| Dressing | (mm) | deviation |
| Batch 1 | 0 | 0 |
| Batch 2 | 8.1 | 1.0 |
| Batch 3 | 74 | 0.4 |

### Low Iodine Loadings

Three different batches of dressing samples were prepared as for the standard iodine loading tests above. Batch 3 represented samples of the invention and batches 1 and 2 were controls.

Masses of molecular iodine and potassium iodide added per sample are displayed in Table 6 below.

TABLE 6

| | Batch 1 | Batch 2 | Batch 3 |
|---|---|---|---|
| | sample | sample | sample |
| Mass of molecular | 0 | 0 | 2.0 |
| iodine (mg) | | | |
| Mass of potassium | 0 | 1.3 | 1.3 |
| iodide (mg) | | | |

Zone on inhibition (ZOI) assays were performed on the following bacteria.

P. aeruginosa

The ZOI assay conducted on P. aeruginosa clearly showed that the iodine had antimicrobial activity. There was a clear zone of inhibition for batch 3 samples of the invention, but no iodine release was visible. The batch 1 and 2 control samples showed no zone of inhibition.

S. aureus

The ZOI assay conducted on S. aureus showed a strong, clear zone of inhibition for batch 3 samples of the invention, although this was smaller than for batch 3 of the standard iodine loading samples. Once again, no iodine release was visible. The batch 1 and 2 control samples showed no zone of inhibition.

The above embodiments are described by way of example only. Many variations are possible without departing from the scope of the invention as defined in the appended claims.

1. An antimicrobial wound dressing comprising a wound dressing substrate comprising at least one species of Formula (I), wherein X is a halogen, and wherein the species is complexed with a non-polymeric counterion and/or complexed with the wound dressing substrate.
[I—I—X]⁻ Formula (I)

2. An antimicrobial wound dressing as claimed in claim 1, wherein the non-polymeric counterion is independently chosen from: an alkali metal cation, an alkaline earth metal cation, a group III metal cation, a transition metal cation, an ammonium cation, an aromatic nitrogen-based cation, and combinations thereof.

3. An antimicrobial wound dressing as claimed in any preceding claim, wherein the species is complexed with a non-polymeric counterion and with the wound dressing substrate.

4. An antimicrobial wound dressing as claimed in any preceding claim, wherein the species of Formula (I) is complexed with the wound dressing substrate by an ionic interaction.

5. An antimicrobial wound dressing as claimed in any preceding claim, wherein the species is complexed with the wound dressing substrate via a counterion that is chemically associated with the wound dressing substrate.

6. An antimicrobial wound dressing as claimed in any preceding claim, wherein the species is complexed with the wound dressing substrate via at least one cationic group of the substrate.

7. An antimicrobial wound dressing as claimed in any preceding claim, wherein the wound dressing substrate is formed from at least one polymer, preferably a fibrous polymer.

8. An antimicrobial wound dressing as claimed in claim 7, wherein the at least one polymer comprises at least one ionic polymer, preferably at least one anionic polymer, preferably having at least one carboxyl moiety.

9. An antimicrobial wound dressing as claimed in claim 8, wherein the at least one polymer comprises a cellulosic polymer comprising a carboxyalkyl cellulose or derivative thereof.

10. An antimicrobial wound dressing as claimed in claim 9, wherein the wound dressing substrate is formed from at least one carboxymethylcellulose polymer having a degree of substitution of between 0.05-0.5.

11. An antimicrobial wound dressing as claimed in claim 9, wherein the wound dressing substrate is formed from at least one carboxymethylcellulose polymer having a degree of substitution of greater than 0.5.

12. An antimicrobial wound dressing as claimed in any one of claims 7 to 11, wherein the at least one species of Formula (I) is held within the polymer structure, preferably within the three-dimensional structure of the polymer.

13. An antimicrobial wound dressing as claimed in any preceding claim, wherein X is a halogen that is independently chosen from: fluorine, chlorine, bromine, and iodine, preferably wherein X is iodine and the at least one species of Formula (I) comprises triiodide.

14. An antimicrobial wound dressing as claimed in any preceding claim, wherein the at least one species of Formula (I) is present in a total amount of between 0.05-20 wt. % of the wound dressing substrate.

15. An antimicrobial wound dressing as claimed in any preceding claim, wherein the wound dressing substrate comprises at least one interstitial region, and at least one species of Formula (I) is retained therein.

16. An antimicrobial wound dressing as claimed in any preceding claim, wherein the wound dressing has a releasable iodine content of between 0.5-18 wt. % of the wound dressing substrate.

17. An antimicrobial wound dressing as claimed in any preceding claim, wherein at least one species of Formula (I) is present at and/or on a surface of the wound dressing substrate at a surface density of between 0.25-8 mg/cm².

18. A method of preparing an antimicrobial wound dressing comprising the steps of:
a. Providing a wound dressing substrate;
b. Dissolving molecular iodine and at least one halide-containing species in a solvent to provide a solution; and
c. Treating the wound dressing substrate with the solution to provide the antimicrobial wound dressing.

19. A method as claimed in claim 18, wherein the solvent in step (b) comprises water and a water-miscible polar organic solvent, preferably wherein the amount by volume of the water-miscible polar organic solvent is greater than or equal to the amount by volume of water in the solvent.

20. A method as claimed in claim 19, wherein the water and water-miscible polar organic solvent are present in a water:polar organic solvent ratio of between 5:95 and 50:50 vol.:vol.

21. A method of preparing an antimicrobial wound dressing comprising the steps of:
a. Providing a wound dressing substrate;
b. Dissolving molecular iodine in a solvent comprising water and a water-miscible polar organic solvent in a water:polar organic solvent ratio of between 5:95 and 50:50 vol.:vol. to provide a solution; and
c. Treating the wound dressing substrate with the solution to provide the antimicrobial wound dressing.

22. A method as claimed in claim 21, wherein step (b) comprises dissolving the molecular iodine and at least one halide-containing species in the solvent.

23. A method as claimed in any one of claims 18 to 20 and 21, wherein the at least one halide-containing species comprises at least one iodide-containing species.

24. A method as claimed in any one of claims 20 to 23, wherein the solvent in step (b) comprises water and a water-miscible polar organic solvent in a water:polar organic solvent ratio of between 10:90 and 30:70 vol.:vol., preferably of between 15:85 and 25:75 vol.:vol.

25. A method as claimed in any one of claims 19 to 24, wherein the solvent in step (b) comprises water and a water-miscible polar protic organic solvent, wherein the water-miscible polar protic organic solvent preferably comprising at least one solvent that is independently chosen from: a carboxylic acid, a phenol, an alcohol, and combinations thereof.

26. A method as claimed in claim 25, wherein the water-miscible polar protic organic solvent comprises at least one C1-C10, preferably C1-C5 alcohol.

27. A method as claimed in any one of claims 18 to 26, wherein the solution prepared in step (b) has a pH of between 4-10.

28. An antimicrobial wound dressing obtainable by the method as claimed in any one of claims 18 to 27.
